(19) United States
(12) Patent Application Publication
(10) Pub. No.: US 2025/0208089 A1
(43) Pub. Date: Jun. 26, 2025

(54) DEVICES AND METHODS FOR RAPID BIOSENSING

(71) Applicant: New York University, New York, NY (US)

(72) Inventors: Elisa Riedo, Tenafly, NJ (US); Davood Shahrjerdi, New York, NY (US); Zhujun Huang, Upton, NY (US); Moeid Jamalzadeh, Brooklyn, NY (US); Bayan Nasri, New York, NY (US)

(21) Appl. No.: 18/848,241

(22) PCT Filed: Mar. 20, 2023
(86) PCT No.: PCT/US2023/064695

§ 371 Date: Sep. 18, 2024

Related U.S. Application Data
(60) Provisional application No. 63/321,337, filed on Mar. 18, 2022.

(51) Int. Cl. G01N 27/414; G01N 27/416

(52) U.S. Cl. CPC G01N 27/4145; G01N 27/4148; G01N 27/4163

(57) ABSTRACT

The present invention relates to a method of measuring a concentration of a target analyte in a volume, comprising the steps of providing a biosensor; exposing the biosensor to the volume; periodically measuring a sensor response; calculating a slope of the measured sensor response as the analytes bind to the capturing probes on the surface; and calculating the concentration of the target analyte in the volume based on the slope of the measured sensor response. The invention also relates to a system for measuring a concentration of a target analyte in a volume, the system comprising: a biosensor having a sensing surface and an output terminal, the output terminal configured to change electrical signal based on the response from the sensing surface; and a pulse shaping detection circuit configured to increase temporal resolution of sensing and to reduce noise in the sensor response during a binding phase.

### CROSS-REFERENCE TO RELATED APPLICATIONS

This application claims to and the benefit of the U.S. Provisional Application No. 63/321,337, filed Mar. 18, 2022, the disclosure of which is incorporated herein by reference in its entirety.

### BACKGROUND OF THE INVENTION

Development of new sensing paradigms underpins many exciting discoveries in life sciences. One objective of emergent sensing technologies is to enable high-throughput screening of biochemical reactions with enhanced detection accuracy at lower cost. Among various emerging technologies, the pioneering work of Bergveld revealed a new direction for massively parallel detection of biochemical reactions using silicon field-effect transistors (FETs)—the building block of modern integrated circuits. In fact, conventional bulk silicon transistors are now used commercially for biosensing. Further, the evolution of transistor technology to use nanoscale channel materials will directly benefit biochemical FET sensors (bioFETs) at a fundamental level. Specifically, many studies have demonstrated improved bioFET sensor response to external charges by increasing the surface-to volume ratio of the channel material using nanowire, nanotube, and nanoribbon structures. These bioFETs can detect a variety of biochemical reactions with concentrations below a few picomolar.

While there have been significant advances in development of electronic and optical sensors, one of the major issues remains the capability to functionalize the electronic active region such that the target molecule/biomolecule can be (i) selectively attached to the desired active region, and (ii) the spatial density distribution can be controlled and it is consistent among all sensors in the sensing platform, both in a reproducible way. Furthermore, a universal methodology to achieve this functionalization across different sensing platforms, devices, and materials is currently lacking.

In a related aspect, biosensing is typically performed in two modalities. The first is electrical, for example the Ion Torrent DNA sensing platform. The second is optical sensing, for example Illumina. There is a significant interest in the field to reduce the cost of the sensing platform and also to reduce the detection and processing time.

### SUMMARY OF THE INVENTION

In one aspect, the present invention relates to a method of measuring a concentration of a target analyte in a volume, comprising: providing a biosensor; exposing the biosensor to the volume; periodically measuring a sensor response; calculating a slope of the measured sensor response as the analytes bind to the capturing probes on the surface; and calculating the concentration of the target analyte in the volume based on the slope of the measured sensor response. In one embodiment, the biosensor comprises a sensor surface and at least one surface capturing probe. In one embodiment, the step of calculating a slope of the measured sensor response comprises calculating a slope of the measured sensor response during the binding phase a plurality of times, and averaging the calculated slope. In one embodiment, the biosensor comprises a pulse shaping circuit configured to increase the temporal resolution of the sensor. In one embodiment, the biosensor comprises a pulse shaping circuit configured to reduce noise in the sensor response during the binding phase. In one embodiment, the pulse shaping circuit comprises a (CR)ⁿ-(RC)^{m} circuit. In one embodiment, the (CR)ⁿ-(RC)^{m} circuit is configured for use in pulse-shaping of signals with time constants of 1 to 1000 sec⁻¹. In one embodiment, the (CR)ⁿ-(RC)^{m} circuit comprises a (CR)²—(RC)² circuit. In one embodiment, step of calculating a slope of the measured sensor response comprises calibrating based on the output of a pulse shaping detection circuitry. In one embodiment, the method further comprises the step of calibrating the biosensor for quantifying association and dissociation rate constants.

In one aspect, the present invention relates to a system for measuring a concentration of a target analyte in a volume, comprising: a biosensor having a sensing surface and an output terminal, the output terminal configured to change electrical signal based on the response from the sensing surface; and a pulse shaping detection circuit configured to increase temporal resolution of sensing and to reduce noise in the sensor response during a binding phase. In one embodiment, the pulse shaping detection circuit comprises two first order high-pass filters connected in a series having an input and an output, having the input of the series electrically connected to the output terminal. In one embodiment, the pulse shaping detection circuit comprises a second order low-pass filter having an input and an output, the input of the second order low-pass filter connected to the output of the series. In one embodiment, the pulse shaping detection circuit comprises a gain stage having an input and an output, the input of the gain stage connected to the output of the second order low-pass filter. In one embodiment, the pulse shaping detection circuit comprises a (CR)ⁿ-(RC)^{m} circuit. In one embodiment, the (CR)ⁿ—(RC)^{m} circuit is configured for use in pulse-shaping of signals with time constants of 1 to 1000 sec⁻¹. In one embodiment, the (CR)ⁿ-(RC)^{m} circuit comprises a (CR)²—(RC)² circuit. In one embodiment, the pulse shaping detection circuit comprises a plurality of amplification stages along a signal chain. In one embodiment, an electrical signal output of the pulse shaping detection circuit is the predictor of the analyte concentration. In one embodiment, the amplitude of the output of the pulse shaping detection circuit is proportional to the slope of the electrical output signal of the front-end biosensing stage.

In one embodiment, a front-end sensing stage of the pulse shaping detection circuit comprises a differential amplifier pair of biosensors. In one embodiment, the output signal of the front-end sensing stage feeds a signal chain of the pulse shaping detection circuit. In one embodiment, one biosensor of the differential amplifier pair has capturing probes on its surface. In one embodiment, one biosensor of the differential amplifier pair does not have capturing probes on its surface. In one embodiment, the differential amplifier pair of biosensors cancels the electrical signals due to non-specific binding. In one embodiment, the differential amplifier pair of biosensors cancels the environmental noise. In one embodiment, the front-end biosensing stage is a single-stage amplifier. In one embodiment, the output of the front-end biosensing stage feeds a signal chain of the pulse shaping circuitry. In one embodiment, the biosensor of the front-end amplifier has capturing probes on its surface.

In one aspect, the present invention relates to a device comprising a functionalized biosensor for detecting the presence of an analyte comprising: a field effect transistor (FET) coated with an external stimulus-responsive polymer layer, wherein the external stimulus-responsive polymer layer is configured to be locally modified via a localized external stimulus to expose, in a desired sensing area, a chemical functional group that is configured to attach to a capture-molecule. In one embodiment, the heat-responsive polymer comprises a first functional group and a second functional group, in which the external stimulus removes the first functional group from the polymer such that the second activated functional group is exposed. In one embodiment, the first functional group comprises at least one of tetrahydropyranyl carbamate, amine N-oxide, tetrahydropyranyl ether, triphenylmethyl ether, tetrahydropyranyl carbonate ester, S-tetrahydropyranyl carbonyl, ethyl disulfide, cyclopropenone, and tertiary butyl ester groups, and wherein the second activated functional group comprises at least one of an amine, alcohol, phenol, or thiol. In one embodiment, the second functional group is an amine. In one embodiment, the polymer is produced by reversible addition fragmentation (RAFT) polymerization, atom transfer radical polymerization (ATRP), or nitroxide mediated radical polymerization.

In one embodiment, the polymer is represented by a formula Aₘ-Bₙ; wherein m, and n are independently positive integers; A is a monomer residue comprising a cross-linking functional group; B comprises a monomer residue with a protected functional group that can be deprotected by external stimulus; and A and B are each connected to a polymer backbone. In one embodiment, the polymer is represented by a formula Aₘ-Bₙ-Cₒ; wherein m, n and o are independently positive integers; A is a monomer residue comprising a cross-linking functional group; B comprises a monomer residue with a protected functional group that can be deprotected by external stimulus; and C is a monomer residue which comprises a solubilizing group; wherein A, B, and C are each connected to a polymer backbone. In one embodiment, A comprises cinnamate methyl ester. In one embodiment, B comprises at least one of tetrahydropyranyl carbamate, amine N-oxide, tetrahydropyranyl ether, triphenylmethyl ether, tetrahydropyranyl carbonate ester, S-tetrahydropyranyl carbonyl, ethyl disulfide, cyclopropenone, and tertiary butyl ester groups. In one embodiment, C comprises an alkyl, alkoxy, or aryl chain. In one embodiment, the polymer backbone comprises one or more polymers selected from the group consisting of poly(methacrylate), poly(acrylate), poly(ester), poly(styrene), poly(amide), poly(olefin), and combinations, co-polymers, statistical co-polymers, gradient co-polymers, or block co-polymers thereof.

In one embodiment, the external stimulus is heat and the polymer is heat-responsive. In one embodiment, the functional group is produced or activated upon local heating of a heat-responsive polymer. In one embodiment, the localized external stimulus comprises electromagnetic radiation. In one embodiment, the exposed functional group comprises a functional group selected from the group consisting of a thiol, an alcohol, a carboxylic acid and its derivatives, an amine, an alkyne, an aldehyde and a ketone. In one embodiment, the exposed functional group is further converted to an alkyne, azide, cycloalkyne, cyclopropenone group. In one embodiment, the exposed functional group is configured using a click chemistry reaction selected from the group consisting of thiol-ene, thiol-yne, copper catalyzed alkyne-azide cycloaddition, strain promoted alkyne-azide cycloaddition, sulfur-fluoride exchange, and Diels-Alder reaction to attach the desired capture-molecule. In one embodiment, the exposed functional group is configured to attach the desired capture-molecule via 1,3-cycloaddition of di-azides and di-ynes to form poly(arylenetriazolylene). In one embodiment, the exposed functional group is configured to attach the desired capture-molecule via click reactions of azides and alkynes to form polymers with 1,2,3-triazole functionalities. In one embodiment, the capture-molecule is selected from the group consisting of an aptamer, antibody, antibody fragment, oligonucleotide, peptide, enzyme, nanobody, and small molecule.

In one embodiment, the device further comprises a second FET that does not comprise a capture-molecule; wherein the second FET is configured for differential detection. In one embodiment, the device is functionalized with a plurality of different capture molecules configured in an array; wherein the device is configured for parallel detection of a plurality of analytes. In one embodiment, a scanning probe, a scanning electron beam, or a localized source of light is used to apply a localized electromagnetic field to the polymer layer. In one embodiment, functional groups or capture-molecules are immobilized on the polymer or a FET sensing region by printing methods. In one embodiment, a second polymer is deposited on top of the external stimulus-responsive polymer layer, wherein the second polymer is configured as an anti-fouling coating to reduce non-specific bindings of capture molecules outside a sensing region. In one embodiment, the second polymer is removed by the localized external stimulus to expose the external stimulus-responsive polymer layer. In one embodiment, a sensing electrode is integrated on the same chip as the FET. In one embodiment, a sensing electrode is located on a different chip than the FET; wherein the sensing electrode is configured as an extended gate. In one embodiment, detection circuitry is integrated with the FET. In one embodiment, detection circuitry is discrete and manufactured separately from the FET. In one embodiment, the device further comprises detection circuitry integrated within an array of the FET.

In another aspect, the present invention relates to a method of making a biofunctionalized biosensor comprising: providing a transistor comprising a semiconductive layer; coating the transistor with an external stimulus-responsive polymer layer; applying a localized external stimulus to a region of the external stimulus-responsive polymer layer, thereby producing activated functional groups on of the region of the surface of the polymer layer; and exposing the polymer layer to a capture-molecule, wherein the capture-molecule attaches to the activated functional groups of the polymer layer. In one embodiment, the localized external stimulus comprises heat. In one embodiment, the heat is applied using thermal scanning probe lithography (tSPL). In one embodiment, the heat is applied using a focused light or a laser. In one embodiment, the localized external stimulus comprises localized electromagnetic radiation. In one embodiment, the localized electromagnetic radiation is applied using a scanning probe, a scanning electron beam, or a localized source of light.

In one embodiment, the activated functional group comprises an amine group. In one embodiment, the method further comprises the step of functionalizing the amine to create patterns of functional groups selected from the group of amine, amide, ammonium, maleimide, aldehyde, thiol, biotin, alkyne, cycloalkyne, cyclopropenone, alkene, and azide. In one embodiment, the capture molecule is selected from the group consisting of an aptamer, antibody, antibody fragment, oligonucleotide, peptide, enzyme, nanobody, and small molecule. In one embodiment, the polymer is produced by radical polymerization.

In one aspect, the present invention relates to a method of detecting the presence of an analyte of interest comprising: providing a device disclosed herein; obtaining a sample; administering the sample to the device; and detecting a change in the electrical signal of the FET, thereby indicating the presence of a target analyte in the sample. In one embodiment, the target analyte is in a fluid sample. In one embodiment, the target analyte is in an air sample. In one embodiment, the target analyte is selected from the group consisting of proteins, nucleic acids, protein fragments, antigens, antibodies, surface receptors, hormones, growth factors, cells, viral particles, bacteria, secreted compounds, heavy metals, toxins, toxic molecules, explosives, pollutants, and metabolites. In one embodiment, the sample is a sample obtained from a subject. In one embodiment, the sample is an environmental sample.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1, FIG. 2, and FIG. 3 are diagrams of a method of fabricating a sensing chip;

FIG. 4 is an exemplary biosensor;

FIG. 5 is a graph of sensor response over time;

FIG. 6 is a diagram of an exemplary FET sensor (see for example Nature Nanotechnology, volume 7 pages 401-407 (2012), hereby incorporated herein by reference in its entirety);

FIG. 7 shows a graph of DNA+HMGB1 detection over time at a range of concentrations (see for example Nature Nanotechnology, volume 7 pages 401-407 (2012), hereby incorporated herein by reference in its entirety);

FIG. 8 is an exemplary pulse shaping circuit;

FIG. 9A, FIG. 9B, FIG. 9C, FIG. 9D, and FIG. 9E are schematic views of an exemplary pulse shaping circuit;

FIG. 10 is a graph of simulation results;

FIG. 11A is a bulk CMOS sensing platform;

FIG. 11B is an SOI CMOS sensing platform;

FIG. 11C is an FDSOI CMOS sensing platform;

FIG. 12 is an exemplary embodiment featuring airborne detection of COVID-19;

FIG. 13 depicts optical images of BioFunFET extended gate devices where the device is coated with a thermally responsive polymer and the graphene gate has been functionalized with Covid-19 antibodies for RBD spike protein detection;

FIG. 14 depicts experimental results for a BioFunFET with graphene extended gate for RBD spike protein detection.

### DETAILED DESCRIPTION

It is to be understood that the figures and descriptions of the present invention have been simplified to illustrate elements that are relevant for a clear understanding of the present invention, while eliminating, for the purpose of clarity, many other elements found in related systems and methods. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

The term “antibody” as used herein, refers to an immunoglobulin molecule, which is able to specifically bind to a specific epitope on an antigen. Antibodies can be intact immunoglobulins derived from natural sources or from recombinant sources and can be immunoactive portions of intact immunoglobulins. Antibodies are typically tetramers of immunoglobulin molecules. The antibodies in the present invention may exist in a variety of forms including, for example, polyclonal antibodies, monoclonals antibodies, Fv, Fab and F(ab)2, as well as single chain antibodies and humanized antibodies (Harlow et al., 1988; Houston et al., 1988; Bird et al., 1988).

The term “analyte” refers to a substance to be detected or assayed by the method of the invention. Typical analytes may include, but are not limited to proteins, peptides, nucleic acid segments, molecules, cells, microorganisms and fragments and products thereof, or any substance for which attachment sites, binding members or receptors (such as antibodies) can be developed.

“Biological sample” as used herein means a biological material isolated from an individual. The biological sample may contain any biological material suitable for detecting a target analyte, and may comprise cellular and/or non-cellular material obtained from the individual.

As used herein, the term “diagnosis” means detecting a disease or disorder or determining the stage or degree of a disease or disorder. Usually, a diagnosis of a disease or disorder is based on the evaluation of one or more factors and/or symptoms that are indicative of the disease. That is, a diagnosis can be made based on the presence, absence or amount of a factor which is indicative of presence or absence of the disease or condition. Each factor or symptom that is considered to be indicative for the diagnosis of a particular disease does not need be exclusively related to the particular disease, i.e. there may be differential diagnoses that can be inferred from a diagnostic factor or symptom. Likewise, there may be instances where a factor or symptom that is indicative of a particular disease is present in an individual that does not have the particular disease. The diagnostic methods may be used independently, or in combination with other diagnosing and/or staging methods known in the medical art for a particular disease or disorder.

The “level” of one or more target analyte means the absolute or relative amount or concentration of the analyte in the sample.

“Measuring” or “measurement,” or alternatively “detecting” or “detection,” means assessing the presence, absence, quantity or amount (which can be an effective amount) of a given substance within a sample, including the derivation of qualitative or quantitative concentration levels of such substances.

“Standard control value” as used herein refers to a predetermined amount of a particular analyte that is detectable in a sample. The standard control value is suitable for the use of a method of the present invention, in order for comparing the amount of a target analyte of interest that is present in a sample. An established sample serving as a standard control provides an average amount of the analyte of interest in the sample type (e.g., biological sample) that is typical for an average, healthy person of reasonably matched background, e.g., gender, age, ethnicity, and medical history. A standard control value may vary depending on the target analyte of interest and the nature of the sample.

The terms “subject,” “patient,” “individual,” and the like are used interchangeably herein, and refer to any animal, or cells thereof whether in vitro or in situ, amenable to the methods described herein. In certain non-limiting embodiments, the patient, subject or individual is a human.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 and any whole and partial increments therebetween. This applies regardless of the breadth of the range.

Biofunctionalized Field-Effect Transistor (biofunFET)

In one embodiment, the invention relates to systems and devices comprising a biofunctionalized field-effect transistor (biofunFET) and methods of using the same. The present invention improves upon the current state of bioFETs by creating a CMOS compatible biofunFET fabrication platform that can be extended to a large variety of biosensing applications, reducing variability, reducing noise, improving sensibility, reproducibility and parallel detection, and increasing stability and robustness.

The ability to precisely control the position or localization of molecules/biomolecules on a surface is critical for several applications including biosensing, bionanoreactors, and single molecule studies. Despite recent advances, fabrication of functional patterns with resolution at the single molecule level is limited by the lack of lithography methods that combine high resolution, compatibility with soft, polymeric structures, ease of fabrication, and high throughput.

In certain aspects, the biofunFET of the present invention comprises a FET comprising a source electrode, drain electrode, a gate electrode, and a semiconducting material. In one embodiment, the biofunFET comprises a polymer coated upon a FET, where this polymer can be locally activated/patterned and locally functionalized/patterned with any desired chemical molecule. For example, in certain embodiments, the polymer is sensitive to, or responsive to, an external stimulus (e.g., heat), wherein a localized stimulus applied to a specific area of the polymer induces or activates the formation of a functional group (also referred to herein as a “sticky end”) in the desired specific area. In one embodiment, the activated functional group of the polymer, for example an amine group, is used to attach (directly or by using other functionalization intermediate steps) a probe or capture-molecule to the locally thermally activated area of the polymer, thereby functionalizing the biofunFET in the desired location (sensing area), for example the semiconducting region, or the top gate or the extended gate. In the sensing measurement, the probe or capture-molecule then specifically binds/captures an analyte of interest, such that when the analyte binds to the probe or capture-molecule, a detectable change in the electronic signals of the biofunFET is induced, thereby indicating the presence and quantity of the analyte of interest in a sample. Another possibility is the use of an extended gate where the top gate of a FET is externally electrically connected to a gate which is coated with a thermally (or other local stimulus) sensitive polymer and functionalized to sense the presence of analytes.

The “polymer” may be any polymer that can be locally activated by a localized external stimulus, for example but not limited to a local source of heat, electrical field, light, current, pressure, shear force. This activation can be obtained by an external probe (for example a nano-sized atomic force microscopy tip) or a beam (for example a laser beam) or direct local deposition of molecules. The local activation creates on the polymer surface a chemical functional group that can be used to attach a capture-molecule. The activated patterns on the polymer surface can have a lateral size that ranges from a few picometers to several millimeters.

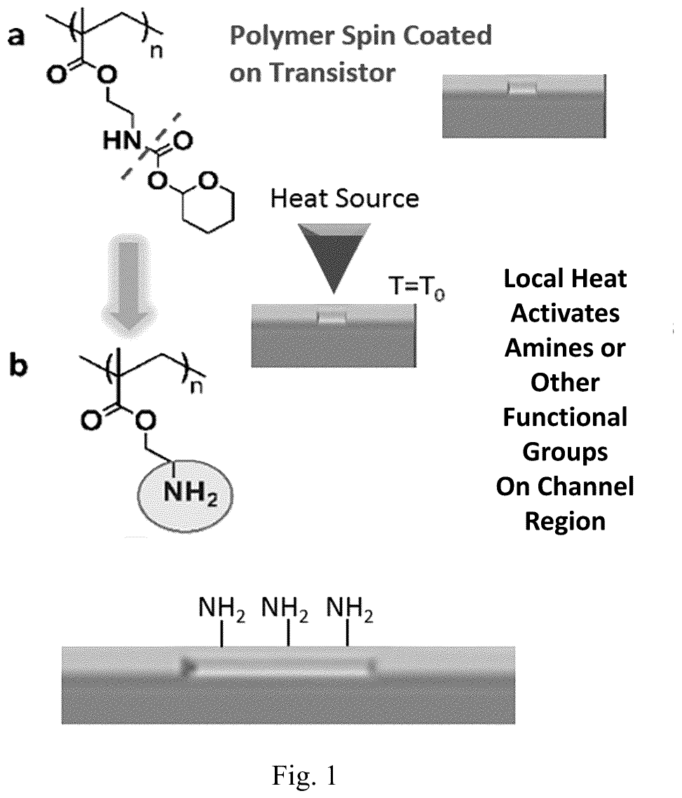

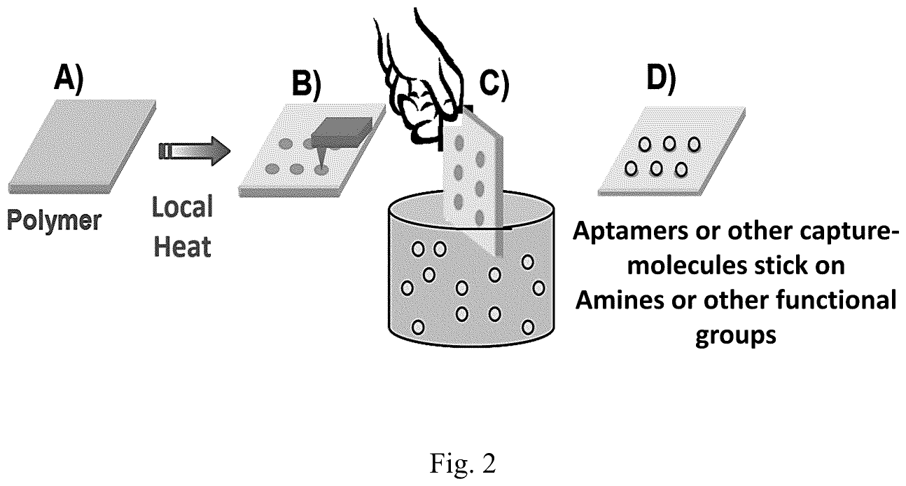

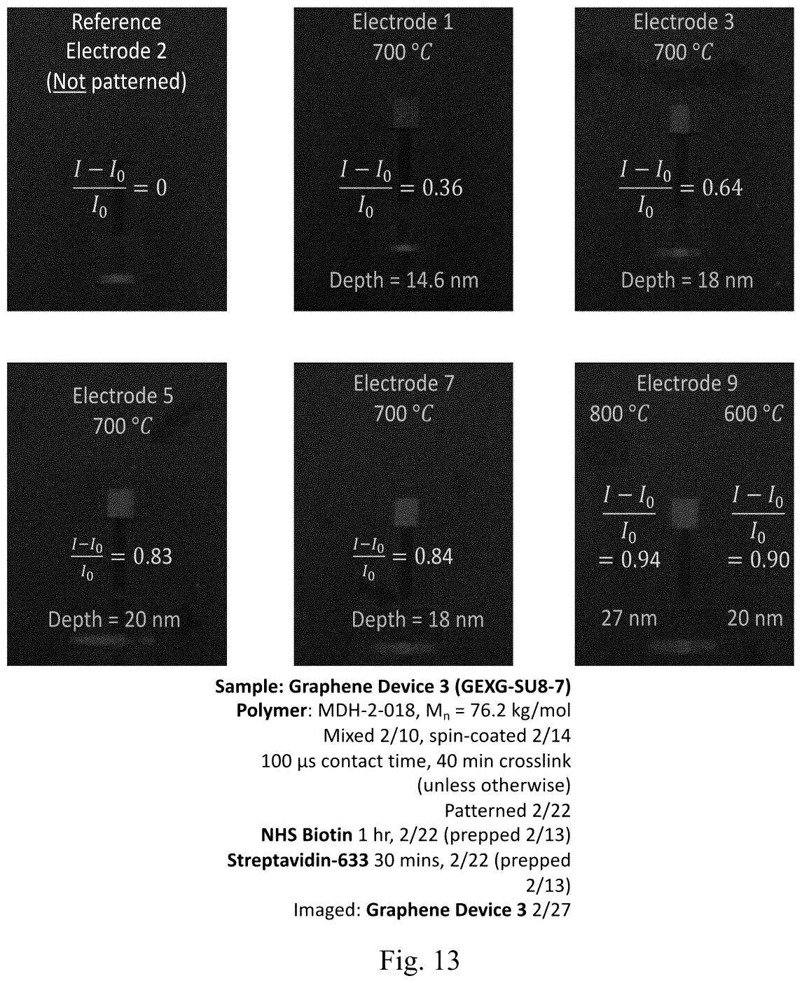

In certain aspects, the ability to generate biofunFETs using local heating of the coated polymer produces biofunFETs with a CMOS compatible biofunFET fabrication platform that can be extended to a large variety of biosensing applications, while reducing variability and noise, improving sensibility, reproducibility and parallel detection, and increasing stability and robustness. For example, in one embodiment, the localized functionalization described in the present invention allows for the generation of a device having a functionalized biofunFET, generated by local heating of the coated polymer as described herein, and a non-functionalized FET right next to the sensor FET, which will not have any capture-molecule on its sensitive area (see FIG. 13). This allows for a differential detection since the device will have a built-in control, wherein signals generated from other sources (other than the analyte), for example non-specific binding of other molecules to the non-functionalized control FET, can be accounted for when measuring/detecting changes in the signals generated from the biofunFET. Further, localized functionalization, as described herein, allows for the production of biofunFETs with various patterns of probes or capture-molecules upon the polymer surface. Further still, localized functionalization allows for the ability to perform parallel detection, where different probes or capture-molecules can be functionalized upon the biofunFETs to allow for the detection of a plurality of analytes within a sample/microchip (see FIG. 1). Since local functionalization by local heat can be achieved with a resolution of 8 nm, the patterns of different capture-molecules produced by local heat can be as small as 10 nm. Importantly, since the biofunFET can be based on a polymer film spin-coated on any electronic device, and local heat, this fabrication platform is fully CMOS compatible, low cost, robust, and reproducible.

In one embodiment, the biofunFET comprises a semiconducting material. Exemplary semiconducting materials of the biofunFET, include, but are not limited to, silicon, carbon nanotubes, III-V and II-VI semiconductors, graphene, 2D materials, MoS₂, transition metal dichalcogenides, or other suitable materials.

In one embodiment, the biofunFET comprises a polymer coated upon the semiconducting material or a gate. In certain embodiments, the polymer comprises a heat-sensitive or heat-responsive polymer. Any polymer in which localized heat or electromagnetic field induces production or activation of a functional group that can be used to attach a probe or capture-molecule, can be used in the present invention. For example, in one embodiment, the polymer comprises a heat-sensitive or heat-responsive polymer, in which an amine group is activated upon local heating. For example, in one embodiment, the polymer comprises a heat-sensitive or heat-responsive polymer, in which the local heat is effective to remove a first functional group from the polymer such that the surface comprises a second functional group at least at a portion of the first location; wherein the first functional group is for example but not limited to a tetrahydropyranyl carbamate, amine N-oxide, tetrahydropyranyl ether, triphenylmethyl ether, tetrahydropyranyl carbonate ester, S-tetrahydropyranyl carbonyl, or ethyl disulfide; and wherein the second functional group is a thiol, an alcohol, a carboxylic acid and its derivatives, an amine, an alkyne, an aldehyde, or a ketone. Tertiary butyl ester groups can also be used as the first functional group followed by localized deprotection. In one embodiment, the tertiary butyl ester group can be converted to a carboxylic acid group with localized external stimulus. In one embodiment, the resulting carboxylic acid may be further converted to an anhydride with additional stimulus (see for example “Atomic force microscopy based thermal lithography of poly(tert-butyl acrylate) block copolymer films for bioconjugation.” Langmuir 24, 10825-10832 (2008); and “Scanning thermal lithography of tailored tert-butyl ester protected carboxylic acid functionalized (Meth)acry-late polymer platforms.” ACS Appl. Mater. Interfaces 3, 3855-3865 (2011)) In other embodiments, the polymer can contain groups that undergo thermal polymerization and cross-linking reactions, including Diels-Alder reactions.

In some embodiments, the polymer comprises a heat-sensitive or heat-responsive polymer, in which an amine group is activated upon local heating. Exemplary polymers that result in activated amines upon local heating include, but are not limited to: poly(methacrylate) copolymer: poly-((tetrahydropyran-2-yl N-(2 methacryloxyethyl) carbamate)-b-(meth-yl 4-(3-methacryloyloxypropoxy) cinnamate)) (PMCC) (see for example “Sub-10 nm Resolution Patterning of Pockets for Enzyme Immobilization with Independent Density and Quasi-3D Topography Control, ACS Applied Materials and Interfaces”, 11, 2019; and “Cost and Time Effective Lithography of Reusable Millimeter Size Bone Tissue Replicas With Sub-15 nm Feature Size on A Biocompatible Polymer”, Adv. Func. Mater. 2021). Also, for example, in some embodiments, the polymer comprises a heat-sensitive or heat-responsive polymer, in which a hydroxyl or carboxylic group is activated upon local heating. Exemplary polymers that result in activated hydroxyl or carboxylic groups upon local heating include, but are not limited to, PMCC at high temperatures (see for example “Sub-10 nm Resolution Patterning of Pockets for Enzyme Immobilization with Independent Density and Quasi-3D Topography Control, ACS Applied Materials and Interfaces”, 11, 2019), and copolymers of benzyl methacrylate and THP-MA, which exhibit thermal deprotection of the THP group to produce carboxylic acid around 150° C., which is followed by conversion to anhydrides above 180° C., a polymer where thermal deprotection of an ester group can produce a carboxylic acid group.

In one embodiment, the exposed amine is further functionalized to create patterns of functional groups selected from the group of amine, amide, ammonium, maleimide, aldehyde, thiol, biotin, and azides.

In some embodiments, the polymer is represented by a formula Aₘ-Bₙ-Cₒ wherein m, n, and o are each independent positive integers, A is a cross-linking functional group, B is a monomer residue with a protected functional group that can be deprotected by external stimulus such as heat or an electromagnetic field, and C is a monomer residue which contains a solubilizing group such as an alkyl, alkoxy, or aryl chain that can increase solubility of the polymeric material. In one embodiment, the bonds between monomer residues A, B, and Care the backbone of the polymer, which is for example but not limited to a poly(methacrylate), poly(acrylate), poly(ester), poly(styrene), poly(amide) or poly(olefin) backbone.

In one embodiment, C is a short alkyl chain. In one embodiment, C is a short alkoxy chain.

In one embodiment, B can be a tetrahydropyranyl carbamate, amine N-oxide, tetrahydropyranyl ether, triphenylmethyl ether, tetrahydropyranyl carbonate ester, S-tetrahydropyranyl carbonyl, or ethyl disulfide. In one embodiment, the different functional group is an amine, alcohol, phenol, or thiol.

In some embodiments, the polymer comprises poly(p-phenylene vinylene) or Poly((tetrahydropyran-2-yl N-(2-methacryloxyethyl) carbamate)-co-(methyl 4-(3-methacryloyloxypropoxy) cinnamate)) as represented by the formula:

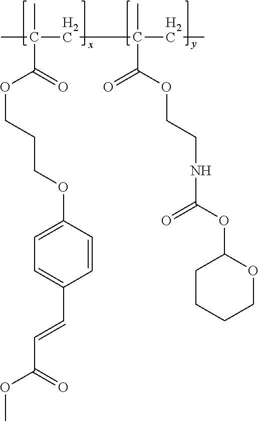

In one embodiment, the polymer is a co-polymer, such that the polymer backbone comprises two or more polymer components. In one embodiment, residues A, B, and C can be on different components of the polymer backbone. In one embodiment, A comprises a cinnamate group. In one embodiment, B comprises a tetrahydropyran carbamate group.

According to some embodiments, the polymer can be represented by a formula Bₙ, wherein B represents a functional group that will be modified by a local external stimulus, n is a positive integer, and the functional group is bound to a polymer backbone. In one embodiment, B is bound to the polymer backbone through a polymer side-chain. In one embodiment, B is part of the polymer backbone. In one embodiment, the polymer comprises more than one polymer backbone and/or functional group. In one embodiment, the polymer can be deposited on the substrate using standard film-forming techniques such as spin-coating, drop casting, blade coating, and spray coating onto a substrate or platform.

In some embodiments, the polymer backbone, can be derived from, or can be, a monomer such as vinyl, allyl, 4-styryl, acryoyl, epoxide, oxetane, cyclic-carbonate, methacryl, acrylonitrile, or the like, which is polymerized by either a radical-, cationic-, atom transfer-, or anionic-polymerization process. In some embodiments, the polymer backbone can be derived from, or can be, an isocyanate, isothiocyanate, or epoxide, that can be copolymerized with di-functional amines or alcohols such as HO(CH₂)_{λ}OH, H₂N(CH₂)_{λ}NH₂, where) is a positive integer (e.g., from 1 to 25). In some embodiments, the polymer backbone can be derived from, or can be, a strained ring olefin (e.g., dicyclopentadienyl, norbornenyl, cyclobutenyl, or the like), which can be polymerized via ring opening metathesis polymerization using an appropriate metal catalyst, as would be known by those skilled in the art to which this disclosure pertains. In some embodiments, the polymer backbone can be derived from, or can be, —(CH₂)ₙSiCl₃, —(CH₂)ₙSi(OCH₂CH₃)₃, or —(CH₂)ₙSi(OCH₃)₃, where the monomers can be reacted with water under conditions known to those skilled in the art to form either thin film or monolithic organically modified sol-gel glasses, or modified silicated surfaces, where n is a positive integer (e.g., from 1 to 25). In some embodiments, the polymer backbone can be derived from, or can be, a polymerizable group that can be photochemically dimerized or polymerized. Such groups can include one or more and these include, but are not limited to, the following conjugated structures:

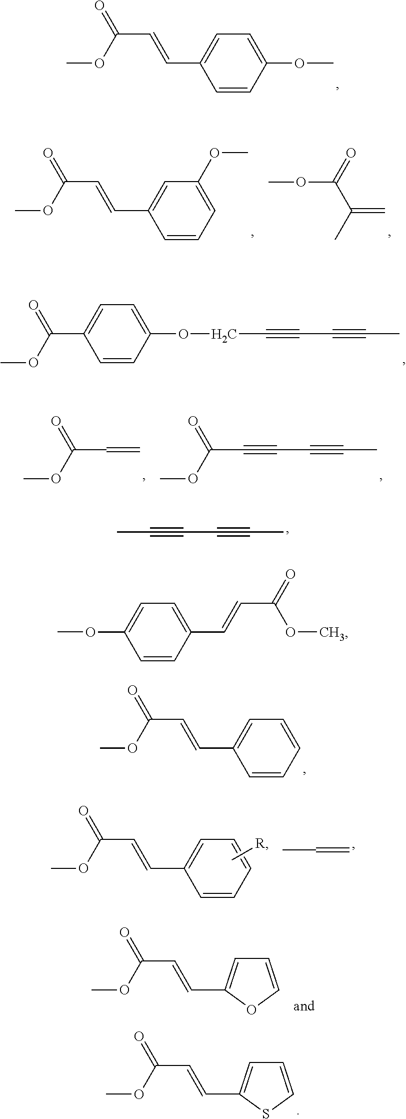

In some embodiments, the functional group, B, can be chosen such that, upon exposure to local external stimuli, a protecting group is removed from the surface, leaving behind another functional group. For example, to obtain a carboxylic acid, B can be chosen form tert-butyl esters, tetrahydropyran esters, and the like. For an amine to result, B can include tetrahydropyranyl carbamates, amine N-oxides, and the like. If an alcohol or phenol is desired, B can be chosen from tetrahydropyranyl ethers, triphenylmethyl ethers, tetrahydropyranyl carbonate esters, and the like. When a thiol is desired exposure to local external stimuli, B can include S-tert-butoxy carbonyls, S-tetrahydropyranyl carbonyls, ethyl disulfides, and the like.

In other cases, B can be a group that undergoes thermal polymerization and cross-linking reactions, including Diels-Alder reactions between two B groups (e.g., furans with maleimides, and the like), ring-opening polymerization (e.g., poly(ferrocenylsilanes) and the like), ring-opening metathesis polymerization (e.g., dicyclopentadiene, and the like), reactions to form conjugated polymers (e.g., from poly(phenylene-vinylene) or other like precursors), and reactions of trifluorovinyl ethers, for example. In some embodiments B can be a group that volatilizes or decomposes from treatment with the local external stimuli.

As stated above, the polymer can have more than one functional group B. These functional groups can be chosen such that each B is modified at the same or a different temperature.

In one embodiment, the polymer can have a group, A, which can be photochemically or thermally cross-linked to control the softening temperature of the overall polymer. In one embodiment, such a polymer may be represented by the formula Aₘ-Bₙ. In one embodiment, with the use of the A group, the softening temperature can be tailored to be above or below the chemical modification temperature as desired. In one embodiment, this can be accomplished by increasing or decreasing the glass transition temperature and/or the crystallinity of the polymer. In one embodiment, the A and B groups can be coupled to the polymer backbone through a side chain, and can be organized in blocks, which can be ordered or randomly oriented. In one embodiment, the A and B groups are derived from the same functional monomer unit. In one embodiment, the A and B groups are derived from different functional monomer units. In some embodiments, the A group can be chosen from cinnamate esters, alkenes, chalcones, trifluorovinyl ethers, Diels-Alder reactants, or the like.

By way of illustration, specific polymers that can be used as the surface for reaction with local external stimuli include the following tetrahydropyran-(THP) protected carboxylic acid-functionalized poly(acrylate):

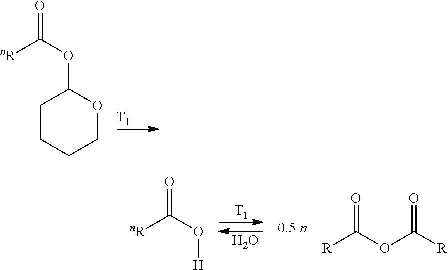

In one embodiment, this type of polymer, which is hydrophobic, can be thermally deprotected at about 120 degrees Celsius (° C.) to give a hydrophilic acid functionality. In one embodiment, the functional group will react further at about 170° C. to give a hydrophobic anhydride. This represents a surface that can undergo a so-called “read-write-overwrite process.” It is important to note that the acid to anhydride conversion is reversible by the removal and addition of water, respectively.

In one embodiment, another polymer that can be used for treatment with local external stimuli is the following poly(amide):

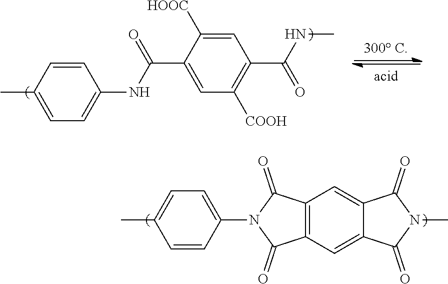

In one embodiment, the starting surface composition (i.e., the poly(amide)) is hydrophilic; but it can be modified into a hydrophobic poly(imide) at about 300° C. In one embodiment, the reaction is reversible with addition of acid.

For example, in one embodiment, the polymer comprises functional groups that can be deprotected by local external stimuli such as heat. In one embodiment, the deprotected functional groups in the activated region of the polymer are then reacted using click-chemistry to attach the desired capture-molecule. In one embodiment, free thiol groups on the surface revealed by the local external stimulus can undergo thiol-ene click chemistry with an alkene-functionalized capture-molecule. In one embodiment, the deprotected amine groups can be converted to azides. In one embodiment, the polymer can incorporate azides as functional groups that are deprotected by external stimuli such as heat. In one embodiment, the polymer can incorporate protected alkenes or alkynes that can be deprotected via local external stimuli to react with an azide-functionalized capture-molecule. In one embodiment, thiols, alcohols, and amines can be turned into alkyne or cycloalkynes for participation in copper-catalyzed and/or strain-promoted alkyne azide click chemistry. In one embodiment, the click chemistry reaction is high yielding without side-reactions, byproducts or the requirement of harsh reaction conditions. In one embodiment, a chemical reaction between the functional group and the desired capture-molecule can be initiated using 1,3-cycloaddition of diazides and diynes to form poly(arylenetriazolylene).

For example, in one embodiment, the polymer comprises two acrylate monomers (a crosslinking monomer containing a cinnamate and a monomer with a protected amine group that can be deprotected under high temperatures) and is formed as a statistical mixture of these two monomers based on feed ratios using a free radical polymerization method. The formation of other polymeric architectures such as block copolymers, gradient copolymers, brush copolymers and blocky copolymers require a living polymerization method. To polymerize the two monomers in a living fashion and to gain access to the polymer architectures described above, reversible addition fragmentation (RAFT) polymerization can be used to polymerize the two monomers in a quasi-living fashion. The livingness of the RAFT polymerization of the two monomers allows for the formation of different polymer architectures. Block copolymers yield materials with defined cross-linking areas and functionalization parts. Additionally, block copolymer from nanostructures on surfaces (such as gyroid and lamellar structures) allowing for predefined special distribution of functional groups. Gradient copolymers allow for the formation of gradient surface functionalization. Blocky copolymers yield smaller superstructures on surfaces with potential new functionalization schemes. Finally, a living polymerization allows for complete control over polymer length, degree of polymerization and polydispersities, all variables that impact surface coverage, crosslinking and film forming properties.

In one embodiment, the polymer is produced by reversible addition fragmentation (RAFT) polymerization, atom transfer radical polymerization (ATRP), or nitroxide mediated radical polymerization.

In some embodiments, another strategy to tune polymeric surface properties is to expand the monomers. A third monomer containing a solubilizing group such as a short alkyl chain will increase solubility of the polymeric material during spin coating. That allows for the use of higher molecular weight polymers to be soluble and spin-coatable. Finally, a third monomer under RAFT polymerization conditions will provide further access to new polymer film forming morphologies and therefore new polymer film properties.

In one embodiment, the surface can be formed from a self-assembled monolayer or multilayer of molecules. The molecules can be represented by the basic structure Xₖ—R—Bₙ, wherein X represents an anchoring group for the molecule to attach to a substrate or platform, R represents a bridging group, C represents the functional group that will be modified by the local external stimulus, and k and n are independently positive integers. In one embodiment, these molecules can be processed by standard self-assembled monolayer- or multilayer-forming techniques, which include a reaction between a thiol-terminated Xₖ—R—Bₙ with a gold surface, silane-terminated Xₖ—R—Bₙ with a glass surface, or like reaction.

In some embodiments, the anchoring group, X, can be chosen from phosphonic acids, phosphinic acids, sulfonic acids, carboxylic acids, carbamates, dithiocarbamates, thiols, selenols, phosphines, amines, amides, carbohydroximic acids, sulfonohydroximic acids, phosphohydroxamic acids, monochlorosilanes, dichlorosilanes, trichlorosilanes, mono(alkoxy) silanes, di(alkoxy) silanes, tri (alkoxy) silanes, or the like, or a conjugate base of any of the foregoing; the bridging group, R, can be a linear or branched C₃ to C₅₀ aliphatic or cyclic aliphatic, fluoroalkyl, oligo (ethyleneglycol), aryl, amine, or like group; and G can be any of the functional group types discussed above for polymeric surfaces.

In one embodiment, the biofunFET comprises a second polymer layer atop a first polymer layer which can help to reduce non-specific binding of the probe/capture-molecule. For example, in one embodiment, the second polymer layer, having low adhesion properties in respect to capture-molecules, is a heat-sensitive or heat-responsive polymer where localized heating evaporates the second polymer layer while also inducing or production or activation of the required functional group in the first polymer layer. Any polymer with these properties can be utilized, for example, but not limited to, is polyphthalaldehyde (PPA). Another example are polymer resists composed of cyclic, low ceiling temperature poly(aldehydes). Another example is molecular glasses. (See for example Microsystems & Nanoengineering (2020)6:21; hereby incorporated herein by reference in its entirety).

In one embodiment, the second polymer is configured as an anti-fouling coating to reduce non-specific bindings of capture molecules outside a sensing region. In one embodiment, the second polymer is removed by the localized external stimulus to expose the external stimulus-responsive polymer layer.

In one embodiment, the biofunFET comprises a probe or capture-molecule attached to the functional group of the heat-sensitive or heat-responsive polymer. The probe or capture-molecule specifically binds to an analyte of interest. Any type of probe or capture-molecules, as known in the art, that can be attached to the biofunFET via the functional group, can be used in the present invention, this includes but it is not limited to antibodies, aptamers and peptides.

The polymer can be selected such that the distance between the probe or capture-molecule and the biofunFET surface is such that association of an analyte of interest with the probe or capture-molecule induces a measurable change in the electronic properties of the biofunFET. In some cases, the polymer is selected such that the distance between the capture-molecule and the surface of the FET is the range of 1 to 300 nm.

In some embodiments, the biofunFET comprises one or more capture-molecules immobilized on the surface of the FET via a linking group, or by direct adsorption to a polymer coating the FET surface. In some embodiments, one or more capture-molecules immobilized on the surface of the FET via a linking group displayed by a polymer coating the FET surface.

In some embodiments the biofunFET comprises a surface coated with a polymer where a local stimulus (such as a local source of heat) generates a free chemical group or “sticky end” such as a free functional amine, hydroxy group, carboxylic group, alcohol, phenol, or thiol or azide. In some embodiments, the “sticky end” functions as an attachment site for attaching one or more capture-molecule for detection of an analyte of interest. In some embodiments the biofunFET comprises a surface coated with a heat sensitive polymer comprising a group which undergoes a heat responsive change (e.g., heat-induced cleavage) such that following heat activation a free chemical group or “sticky end” such as a free functional amine is available. In some embodiments, the “sticky end” functions as an attachment site for attaching one or more capture molecule for detection of an analyte of interest.

In some embodiments the biofunFET comprises a surface coated with a heat sensitive polymer comprising a group which undergoes a heat responsive change (e.g., heat-induced cleavage) such that following heat activation a free chemical group or “sticky end” such as a free functional amine, hydroxyl group, carboxylic group, alcohol, phenol, or thiol or azide. In some embodiments, the “sticky end” functions as an attachment site for attaching one or more capture-molecule for detection of an analyte of interest.

The biofunFET sensors of the invention further include a capture-molecule for an analyte of interest immobilized in proximity to the FET surface, such that association of the analyte of interest with the capture-molecule induces a measurable change in the electrical properties of the FET.

Capture-molecules for particular analytes of interest are known in the art, and can be selected in view of a number of considerations including analyte identity, analyte concentration, and the nature of the sample or sample conditions in which the analyte is to be detected. Suitable capture-molecules include aptamers (nucleic acid or peptide), antibodies, antibody fragments, antibody mimetics (e.g., engineered affinity ligands), peptides (natural or modified peptides), proteins (e.g., recombinant proteins, host proteins), oligonucleotides, DNA, RNA (e.g., microRNAs), and organic small molecules (e.g., haptens or enzymatic co-factors, enzymes).

In some embodiments, the capture-molecules are bound to the activated functional groups of the polymer via biochemical conjugation or electrostatic binding. The binding can occur directly or by using intermediate functional groups, and linkers.

In one embodiment, the capture-molecule of the invention comprises an antibody, or antibody fragment. In certain embodiments, the antibody capture-molecule specifically binds to a compound of interest, for example a secreted compound of interest. Such antibodies include polyclonal antibodies, monoclonal antibodies, Fab and single chain Fv (scFv) fragments thereof, bispecific antibodies, heteroconjugates, human and humanized antibodies.

Such antibodies may be produced in a variety of ways, including hybridoma cultures, recombinant expression in bacteria or mammalian cell cultures, and recombinant expression in transgenic animals. The choice of manufacturing methodology depends on several factors including the antibody structure desired, the importance of carbohydrate moieties on the antibodies, ease of culturing and purification, and cost. Many different antibody structures may be generated using standard expression technology, including full-length antibodies, antibody fragments, such as Fab and Fv fragments, as well as chimeric antibodies comprising components from different species. Antibody fragments of small size, such as Fab and Fv fragments, having no effector functions and limited pharmokinetic activity may be generated in a bacterial expression system. Single chain Fv fragments show low immunogenicity.

In one embodiment, the capture-molecule of the invention comprises an isolated nucleic acid, including for example a DNA oligonucleotide and a RNA oligonucleotide. In certain embodiments, the nucleic acid capture-molecule specifically binds to a compound of interest, for example a DNA molecule or an RNA molecule (e.g., mRNA, rRNA, or lncRNA). The nucleic acid capture probe of the invention may be in the form of a linear oligonucleotide or may have a secondary structure (e.g., a hairpin or loop) which promotes binding and capture of the target analyte.

For example, in one embodiment, the nucleic acid comprises a nucleotide sequence that is complementary to a nucleic acid of interest. The nucleotide sequences of a nucleic acid capture-molecule can alternatively comprise sequence variations with respect to the original nucleotide sequences, for example, substitutions, insertions and/or deletions of one or more nucleotides, with the condition that the resulting nucleic acid functions as the original and specifically binds to the compound of interest.

In one embodiment, the nucleic acid comprises a nucleic acid aptamer. Nucleic acid aptamers are synthetic oligodeoxynucleotides designed according to rigorous recognition and binding affinities between nucleotides, and are obtained by screening through systematic evolution of ligands by exponential enrichments (SELEX). Nucleic acid aptamers not only have features similar to antibodies, such as highly specific recognition and highly binding affinities to targets. An aptamer of the invention can have one or more modified nucleosides or modified nucleobase linkages. For example, in some embodiments, the aptamer may be a thioaptamer that contains one or more phosphorothioate or phosphorodithioate moieties, 2′-fluoro-ribonucleotide oligomers, NH2-substituted and OCH3-substituted ribose aptamers, and deoxyribose aptamers. In some embodiments, the aptamer may be an LNA aptamer.

The term “capture aptamer” as used herein refers to an aptamer that is bound to a substrate (e.g., a functionalized polymer on a biofunFET) and comprises a configuration that can locate (i.e. bind in a sample) a target analyte, thereby causing the target analyte to be attached to the substrate via the capture aptamer upon binding.

The term “aptamers” refers to nucleic acids (typically DNA, RNA or oligonucleotides) that emerge from in vitro selections or other types of aptamer selection procedures well known in the art (e.g. bead-based selection with flow cytometry or high density aptamer arrays) when the nucleic acid is added to mixtures of molecules. Ligands that bind aptamers include but are not limited to small molecules, peptides, proteins, carbohydrates, hormones, sugar, metabolic byproducts, cofactors, drugs and toxins. Aptamers of the invention are specific for a particular target analyte of interest. Aptamers can have diagnostic, target validation and therapeutic applications. The specificity of the binding is defined in terms of the dissociation constant Kd of the aptamer for its ligand. Aptamers can have high affinity with Kd range similar to antibody (pM to nM) and specificity similar/superior to antibody (Tuerk and Gold, 1990, Science, 249:505; Ellington and Szostak, 1990, Nature 346:818). An aptamer will typically be between 10 and 300 nucleotides in length.

Aptamers configured to bind to specific target analytes can be selected, for example, by synthesizing an initial heterogeneous population of oligonucleotides, and then selecting oligonucleotides within the population that bind tightly to a particular target analyte. Once an aptamer that binds to a particular target molecule has been identified, it can be replicated using a variety of techniques known in biological and other arts, for example, by cloning and polymerase chain reaction (PCR) amplification followed by transcription.

In some embodiments, heat activated functionalization of the polymer-coated surface can be temporally restricted, spatially restricted or both spatially and temporally restricted such that only a portion (e.g. a single zone or pattern) of the polymer is functionalized for attaching to a capture-molecule at a given time or in a given location of the surface of the FET (e.g. the channel of the FET or the extended gate). Such an embodiment allows for the generation of multi-probe biofunFET sensors in which different capture-molecules are functionalized in different zones across the surface of the sensor or multi-plex sensors in which different samples can be applied to different zones of the sensor.

Also provided are devices, including probes and multi-well plates, incorporating the biofunFET sensors of the invention.

In one embodiment, the present invention relates to a method of producing a biofunFET, as described herein. In one embodiment, the method comprises coating the semiconducting material of a FET with a heat-sensitive or heat-responsive polymer. For example, in one embodiment, the method comprises spin-coating the semiconducting material of a FET with a heat-sensitive or heat-responsive polymer. Other examples include but are not limited to physical vapor deposition, chemical vapor deposition, sputtering, or other suitable methods.

As described herein, localized heat applied to the polymer induces production or activation of functional groups to which probes or capture-molecules can then be attached. Any sources of localized heat can be used and applied to the polymer surface.

For example, in one embodiment, the method comprises heating or activating the polymer surface with an electromagnetic field, for example lasers, electron beams, electric fields, or other suitable methods.

In one embodiment, the method comprises using thermal scanning probe lithography (tSPL) to locally heat the polymer surface. tSPL (R. Szoszkiewicz, et al., Nano Lett. 2007, 7, 1064) has been proven to be capable of patterning complex quasi-3D topographies on a polymer surface with sub-15 nm lateral resolution and sub-2 nm depth resolution (D. Pires, et al., Science 2010, 328, 732; R. Garcia, et al., Nat. Nanotechnol. 2014, 9, 577; X. Y. Liu, et al., ACS Appl. Mater. Interfaces 2019, 11, 41780). tSPL can be performed using a commercially available instrument, which uses a thermal nanoprobe to locally evaporate the thermosensitive polymer polyphthalaldehyde (PPA), leaving a void that defines the pixel size (S. T. Zimmermann, et al., ACS Appl. Mater. Interfaces 2017, 9, 41454; S. T. Howell, et al., Microsyst. Nanoeng. 2020, 6, 21; X. R. Zheng, et al., Nat. Electron. 2019, 2, 17). In one embodiment, a scanning probe, a scanning electron beam, or a localized source of light is used to apply the localized electromagnetic radiation to the polymer layer.

tSPL can write topographical and chemical features in a thermosensitive polymer resist by local heating, thereby carving and chemically activating the surface with nanoscale precision (X. Y. Liu, et al., ACS Appl. Mater. Interfaces 2019, 11, 41780; D. B. Wang, et al., Adv. Funct. Mater. 2009, 19, 3696). The tSPL process involves first the pixilation of a reference input image, and then the replication of that image by assigning at each grey level of individual pixels a particular height level. Additional information about tSPL and other lithography techniques may be found in U.S. patent application Ser. No. 17/592,169, filed Feb. 3, 2022 and U.S. Pat. No. 8,468,611, issued Jun. 18, 2013, incorporated herein by reference in their entireties.

In one embodiment, the method comprises functionalizing the FET by attaching one or more agents or capture-molecules to the generated or activated functional groups generated by localized heating of the polymer. In one embodiment, the method comprises contacting the surface of the polymer with a liquid medium comprising the probe or capture agent, where the probe or capture agent binds to or otherwise associates with the functional group of the polymer.

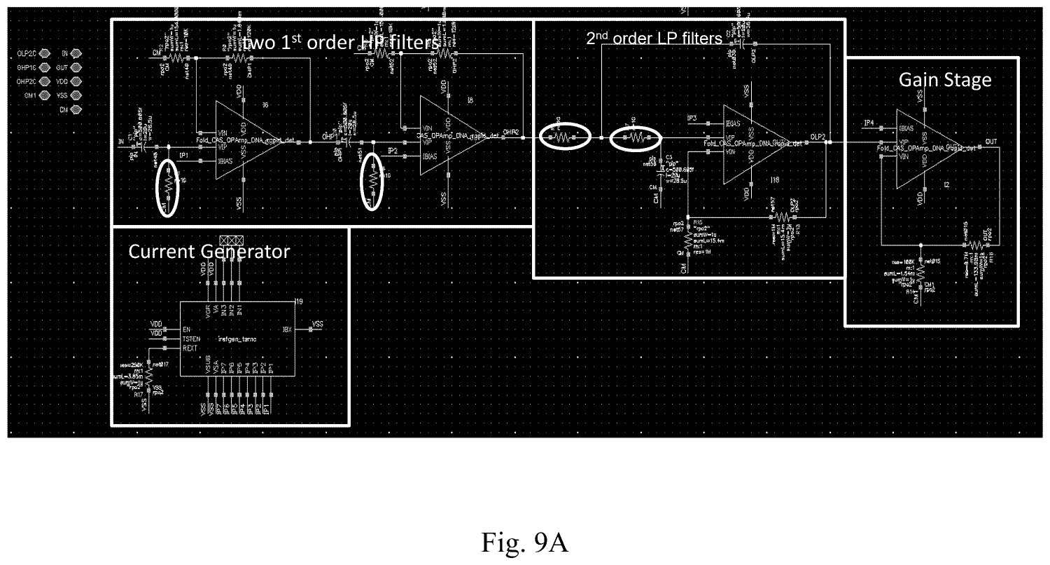

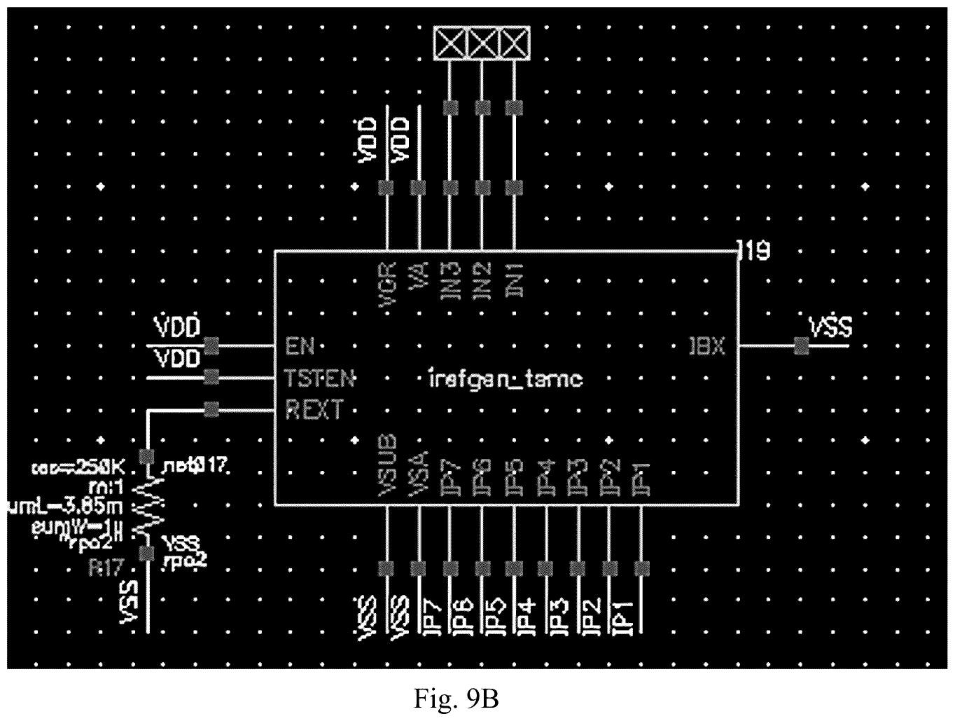

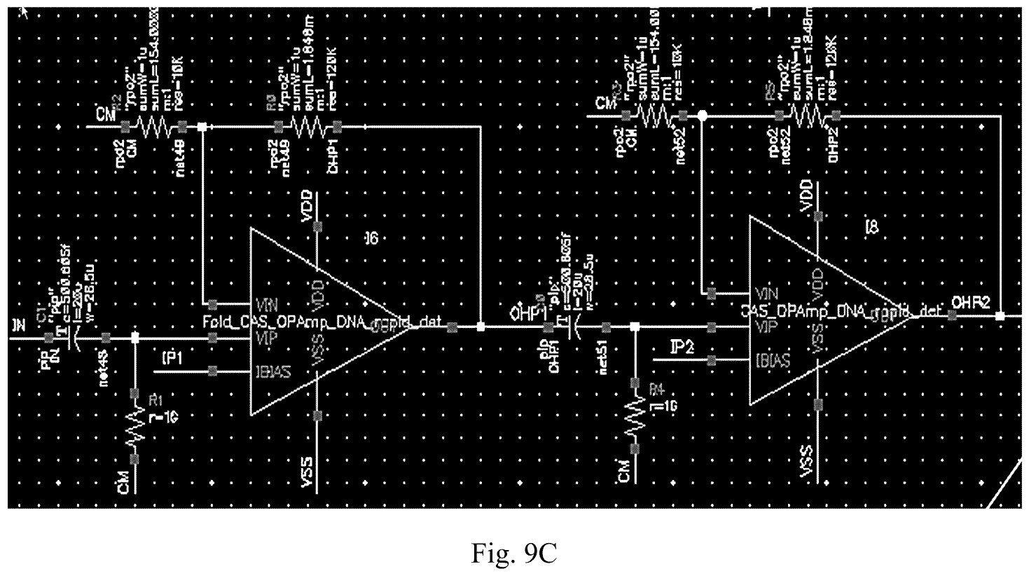

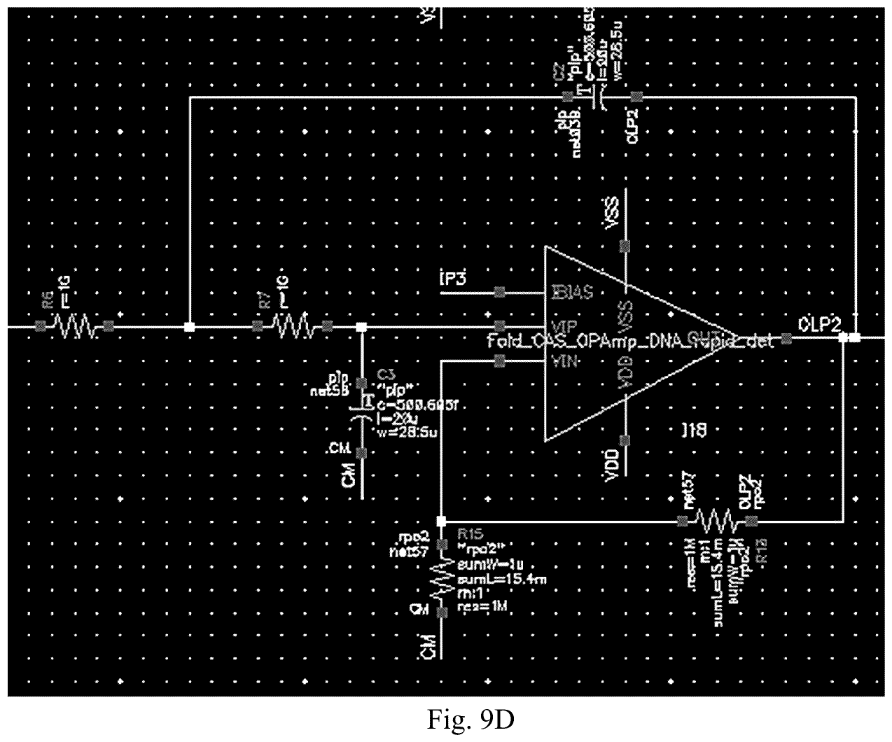

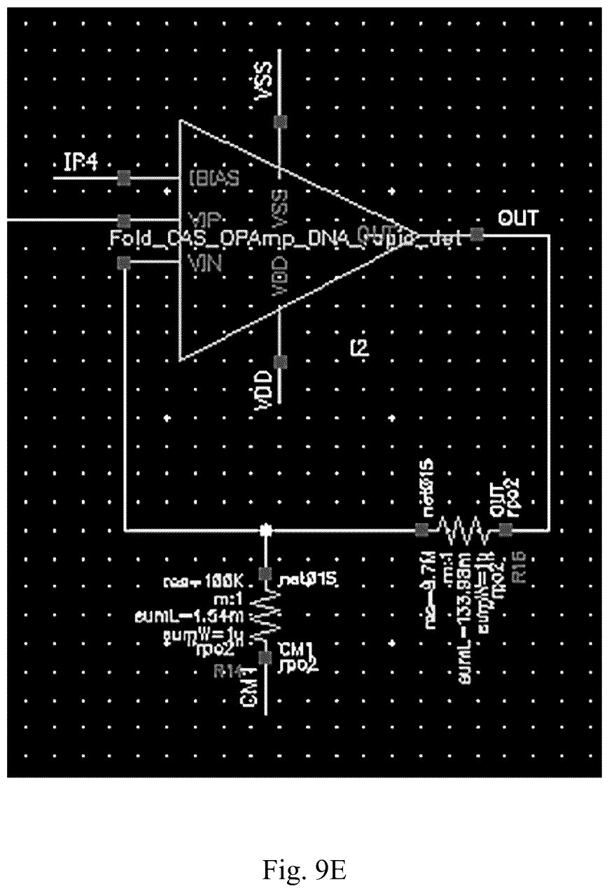

FIG. 9 provides an exemplary methodology of generating the disclosed biofunFET, where a polymer may be spin-coated onto a transistor, where a local heat source is then used to change the surface chemistry and create “sticky” amine nano patterns on the transistor. Aptamers then stick only to the patterned amines. In some embodiments, multimer patterns may be used with different types of aptamers for sensing multiple analytes. In this way, a control FET on the same chip is produced. Another view of the production process is shown in FIG. 2 and in FIG. 3. The steps of FIG. 1 correspond to the steps marked A) and B) in FIG. 2.

Disclosed herein is a new sensing platform that includes the integration of the above tSPL patternable polymers with electronics and optoelectronics devices, which in some embodiments can utilize sensing methods as described above. This integration involves the placement of a polymer on top of a sensor, for example an electronic sensor, a magnetic sensor, and/or an optical sensor, followed by local nanopatterning by local heat, tSPL, nanopatterning means, or other sources of heat (e.g., laser) to functionalize the targeted regions. The novel features of this platform include, but are not limited to, molecules and biomolecules being selectively attached to the desired active region of the sensor post sensor fabrication, for example via patterning of voids or thinner regions of a polymer or other covering material, for example a masking polymer, positioned over a sensor active area.

In some embodiments, the spatial density distribution of molecules on the surface of a sensing platform can be controlled and be made consistent among all sensors in the sensing platform. In some embodiments, arbitrary shaped functionalized patterns with spatial resolution of as low as 10 nm, less than 1 μm, less than 500 nm, less than 200 nm, less than 100 nm, less than 50 nm, less than 20 nm, less than 10 nm, less than 8 nm, less than 5 nm, or less than 3 nm in the sensor active region can be fabricated.

The sensor performance (limit of detection) may in some embodiments be controlled by the thickness of the polymer, where the polymer thickness can be controlled down to a single monolayer, in increments of one, two three, five, or ten monolayers, for example. The polymer thickness can range from 1 nm to 300 nm. In some embodiments, multiple biomolecules can in some embodiments be attached and sensed in the same electronic active region. In some embodiments, the limits of detection can range from femtomolars to nanomolars.

The disclosed platform is a universal methodology to achieve this functionalization across different sensing platforms, devices, and materials.

In some embodiments, disclosed herein is a method of creating a patterned or customized sensing surface, comprising providing a sensor having an active sensing region, positioning or depositing a masking material having a deposited thickness or thickness range, for example a polymer, over the active sensing region, determining a desired sensing surface profile, and lithographically patterning a set of voids or thin regions, the thin regions having a thickness less than the deposited thickness or thickness range in the masking material such that the voids or thin regions form exposed or partially exposed regions, respectively, of the sensing surface configured to act as the desired sensing surface profile. The size of the active sensing region can range from a few 100 nm² to several thousands of mm².

The device and methods of the invention may be used to determine the presence and/or quantity of any target analyte. Suitable types of analytes include proteins, nucleic acids, protein fragments, antigens, antibodies, surface receptors, hormones, growth factors, cells, viral particles, bacteria, secreted compounds, metabolites and the like. The precise combination of compounds of interest being assayed by way of the invention is easily controllable and defined by the eventual user. For example, detection of a particular target analyte is only limited by the availability of a capture agent (e.g. antibody, aptamer, peptide, nucleic acid sequence, etc.) that specifically binds to the compound and can be functionalized to the biofunFET surface. In one embodiment, the capture-molecule comprises an antibody or a capture aptamer, and the target analyte comprises a region or epitope that the antibody or capture aptamer binds to.

In one embodiment, the method of the invention includes the steps of obtaining a sample containing or potentially containing a target analyte, contacting a biofunFET comprising a capture probe for detection of the target analyte of the invention with at least a portion of the sample, detecting a change in electric potential based on the interaction of the target analyte in the sample with the capture probe on the biofunFET, and identifying the sample as containing the target analyte based on the detection of a change in electric potential.

In one aspect, a sample can be contacted with the biofunFET so that a target analyte present in the sample binds with the capture probe on the biofunFET. In one aspect, the present invention relates to methods of detecting the presence or abundance of an analyte of interest using the biofunFET and sensors or devices comprising the biofunFET, as described herein. The present invention can be used to detect the presence or abundance of any analyte of interest. As any probe or capture-molecule can be functionalized using the activated functional groups of the polymer, the probe or capture-molecule can be chosen to specifically bind to the analyte of interest.

The parallel detection ability of the present invention allows for the detection of 2 or more, 3 or more, 4 or more, 5 or more, 10 or more, 20 or more, 40 or more, 50 or more, 100 or more, and the like, target analytes in one or more sample. For example, as is described elsewhere herein, the spatial functionalization of the heat-activated polymer provided by the invention allows for the ability to create distinct zones for parallel capture and detection of analytes.

In certain embodiments, the methods comprise detecting the presence or abundance of an analyte in a sample obtained from a subject, in order to detect the presence or severity of a disease, disorder, or condition in the subject. For example, in certain embodiments, the methods can be used to diagnose the subject as having, or at being at risk for having, a disease, disorder, or condition. In one embodiment, the method is used to determine that the subject has a pathogenic infection (e.g. a viral infection or bacterial infection).

In one embodiment, the invention provides methods of diagnosing a subject as having an infection, or a disease or disorder associated therewith, based on detection of one or more target molecule associated with an infectious agent (e.g., a virus, bacterium, fungus or protozoan). In one embodiment, the invention provides methods of, determining the risk of developing, or assessing progression of an infection, or a disease or disorder associated therewith, by detecting the presence of at least one target molecule in a sample.

In some embodiments, the target analyte comprises a viral particle, including, but not limited to, a viral particle of coronavirus, influenza virus, Zika virus, Ebola virus, Japanese encephalitis virus, mumps virus, measles virus, rabies virus, varicella-zoster, Epstein-Barr virus (HHV-4), cytomegalovirus, herpes simplex virus 1 (HSV-1) and herpes simplex virus 2 (HSV-2), herpes papilloma virus (HPV), human immunodeficiency virus-1 (HIV-1), JC virus, arboviruses, enteroviruses, West Nile virus, dengue virus, poliovirus, and varicella zoster virus. In some embodiments, the target analyte comprises a bacterial marker, including, but not limited to, a marker of Streptococcus pneumoniae, Neisseria meningitides, Streptococcus agalactia, or Escherichia coli. In some embodiments, the target analyte comprises a fungal or protozoan marker, including, but not limited to, a marker of Candidiasis, Aspergillosis, Cryptococcosis, and Toxoplasma gondii.

In one embodiment, the method of the invention is a method of diagnosing a disease or disorder associated with the presence or absence of a target analyte. In one embodiment the method includes the steps of obtaining a sample from a subject having or at risk of having a disease or disorder associated with the presence of a target analyte, contacting a biofunFET comprising a capture probe for detection of the target analyte with at least a portion of the sample, detecting a change in electric potential based on the interaction of the target analyte in the sample with the capture probe on the biofunFET, identifying the sample as containing the target analyte based on the detection of a change in electric potential, and diagnosing the subject as having the disease or disorder associated with the presence of the target analyte. In one embodiment the method includes the steps of obtaining a sample from a subject having or at risk of having a disease or disorder associated with the absence of a target analyte, contacting a biofunFET comprising a capture probe for detection of the target analyte with at least a portion of the sample, detecting a lack of change in electric potential based on the absence of an interaction of target analyte with the capture probe on the biofunFET, identifying the sample as lacking the target analyte based on the detection of a lack of change in electric potential, and diagnosing the subject as having the disease or disorder associated with the absence of the target analyte.

In one embodiment, the method of the invention is a method of treating a disease or disorder associated with the presence or absence of a target analyte. In one embodiment the method includes the steps of obtaining a sample from a subject having or at risk of having a disease or disorder associated with the presence of a target analyte, contacting a biofunFET comprising a capture probe for detection of the target analyte with at least a portion of the sample, detecting a change in electric potential based on the interaction of the target analyte in the sample with the capture probe on the biofunFET, identifying the sample as containing the target analyte based on the detection of a change in electric potential, identifying the subject as having the disease or disorder associated with the presence of the target analyte, and administering a therapeutic agent for the treatment of the disease or disorder associated with the presence of the target analyte. In one embodiment the method includes the steps of obtaining a sample from a subject having or at risk of having a disease or disorder associated with the absence of a target analyte, contacting a biofunFET comprising a capture probe for detection of the target analyte with at least a portion of the sample, detecting a lack of change in electric potential based on the absence of an interaction of target analyte with the capture probe on the biofunFET, identifying the sample as lacking the target analyte based on the detection of a lack of change in electric potential, identifying the subject as having the disease or disorder associated with the absence of the target analyte, and administering a therapeutic agent for the treatment of the disease or disorder associated with the absence of the target analyte.

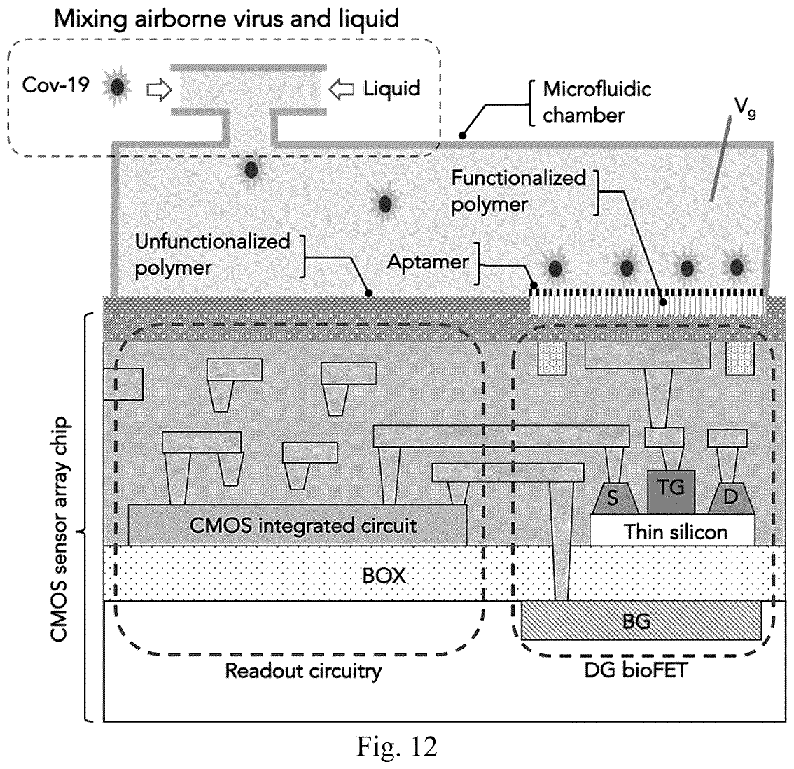

An exemplary embodiment featuring airborne detection of COVID-19 is shown in FIG. 12. In such an embodiment, the air sample suspected to contain a SARS-CoV-2 viral particle is mixed with liquid and applied to a biofunFET sensor of the invention functionalized with an aptamer specific for binding to a SARS-CoV-2 antigen.

Therefore, in one embodiment, the invention provides methods of diagnosing a subject as having a SARS-CoV-2 infection or a disease or disorder associated therewith such as COVID-19. In one embodiment, the invention provides methods of, determining the risk of developing, or assessing progression of a SARS-CoV-2 infection or a disease or disorder associated therewith (e.g., COVID-19) though detection of a SARS-CoV-2 antigen or nucleic acid molecule in a sample.

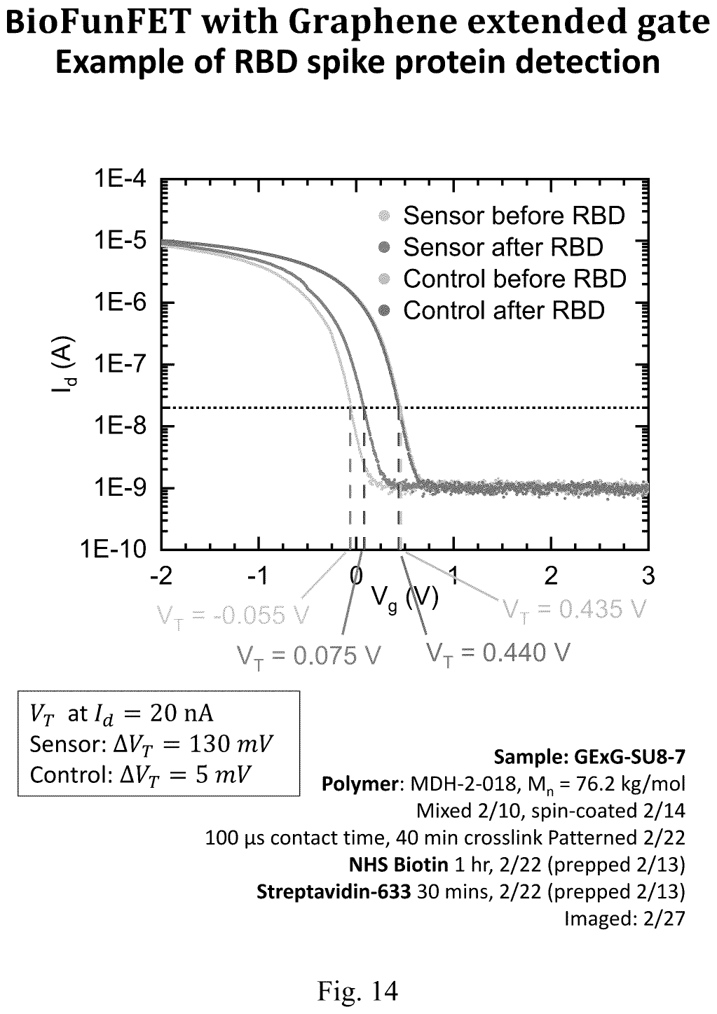

FIG. 13 depicts optical images of BioFunFET extended gate devices where the device is coated with a thermally responsive polymer and the graphene gate has been functionalized with Covid-19 antibodies for RBD spike protein detection. FIG. 14 depicts experimental results for a BioFunFET with graphene extended gate for RBD spike protein detection, which demonstrates a shift in V_{T} after RBD is detected by the sensor.

In certain embodiments, the method comprises detecting the presence or abundance of the analyte in an environmental sample, such as a water sample, sewage sample, air sample, or the like, to determine the presence of the analyte within the environment.

In certain embodiments, the method comprises detecting a change in electrical properties of the biofunFET, wherein a change indicates the presence of the analyte of interest. As described herein, in certain embodiments, the presently described sensors reduce noise by having one functionalized biofunFET and one non-functionalized FET, thereby allowing for a more accurate determination and quantification of analyte presence in a sample. In one specific embodiment, the electrical signals of the biofunFET are analyzed using the rapid sensing methodology described below in further detail. However, the biofunFET and methods of use are not specifically limited to any analysis method.

The disclosed device and methods include three key advantages. The first is biofunctionalization by local heating of ad-hoc polymer spin-coated onto a FET. This provides for a robust chemical functionalization of a biofunFET, allows it to be CMOS (Complementary metal-oxide-semiconductor), FDSOI (Fully Depleted Silicon on Insulator), and DG FDSOI (Double Gate Fully Depleted Silicon on Insulator) compatible (which is advantageous for the microelectronic industry) and allows for local functionalization for different sensing, resulting in a large increase in signal to noise ratio and reproducibility. In some embodiments, the parallel sensing capability may be realized, for example via sequential activation of the polymer, for sensing multiple analytes or viruses on the same biochip. Local heating may be available in CMOS, which allows for localized and on-demand functionalization. DG FDSOI have two independent gates for device-level “capacitive” amplification. In some embodiments, only a portion of the polymer is functionalized, and the non-functionalized portion is utilized as a control.

A second advantage is fast detection using the transient response as disclosed herein, as opposed to steady state measurement methods in which the response time (time to steady state) is dependent on the concentration of the sample being measured. A lower concentration takes longer to reach steady state. A third advantage is that aptamers are cheap, allowing for easier functionalization of biofunFETs and adaptation to new viruses and bacteria, and have strong kinetics for fast detection.

### Rapid Biosensing

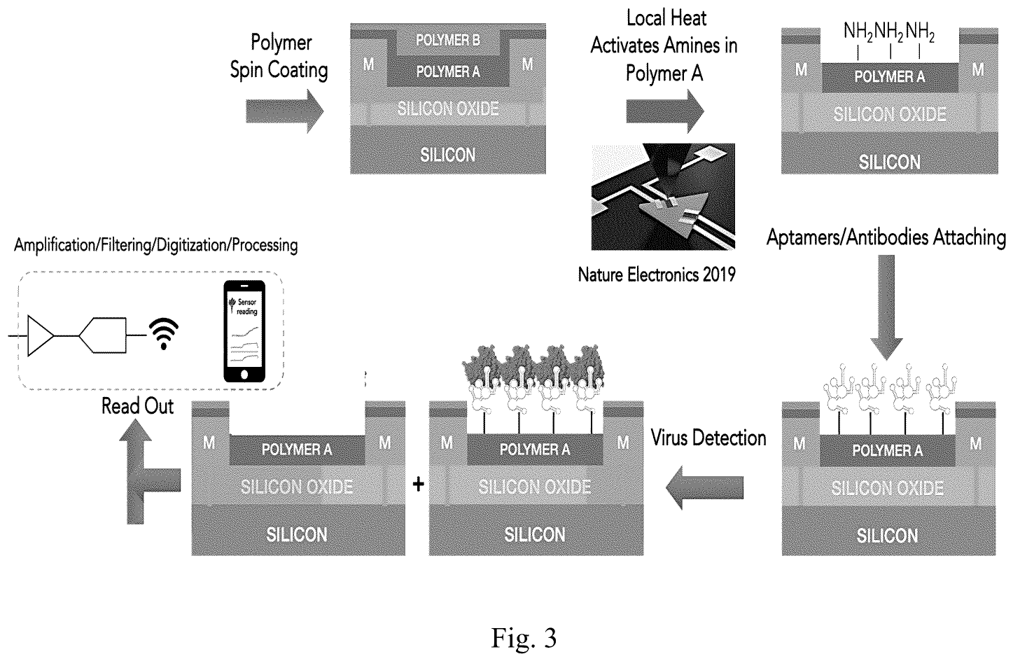

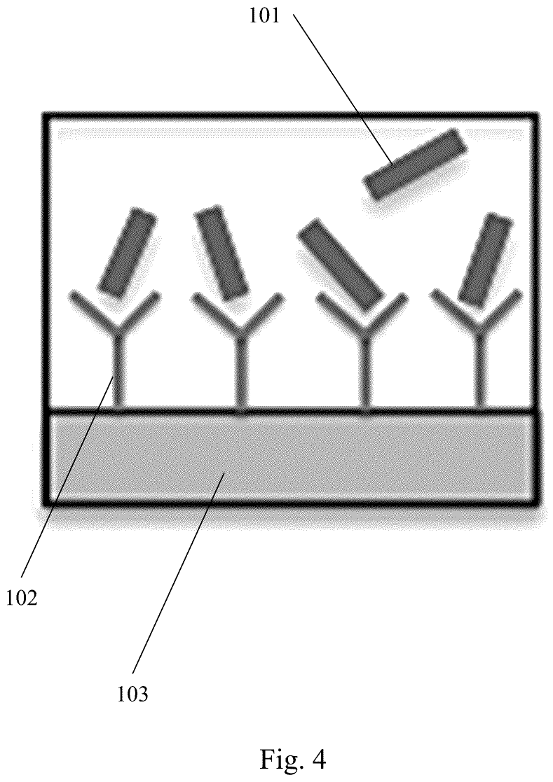

In one aspect, disclosed herein are CMOS biochips and methods for rapid sensing applications. An exemplary biosensor is shown in FIG. 4. The biosensor includes the sensor surface 103 functionalized with one or more capture probes 102, which for detection of at least one target analyte. The target analytes 101 are shown either bound or not bound to the surface probes 102. The target analytes 101 plus the surface probe 102 generates a chemically specific detection. The combination of the target analyte 101 and the surface probe 102 results in a signal generated from the sensor 103. Exemplary target analytes that can be detected include those described elsewhere herein.

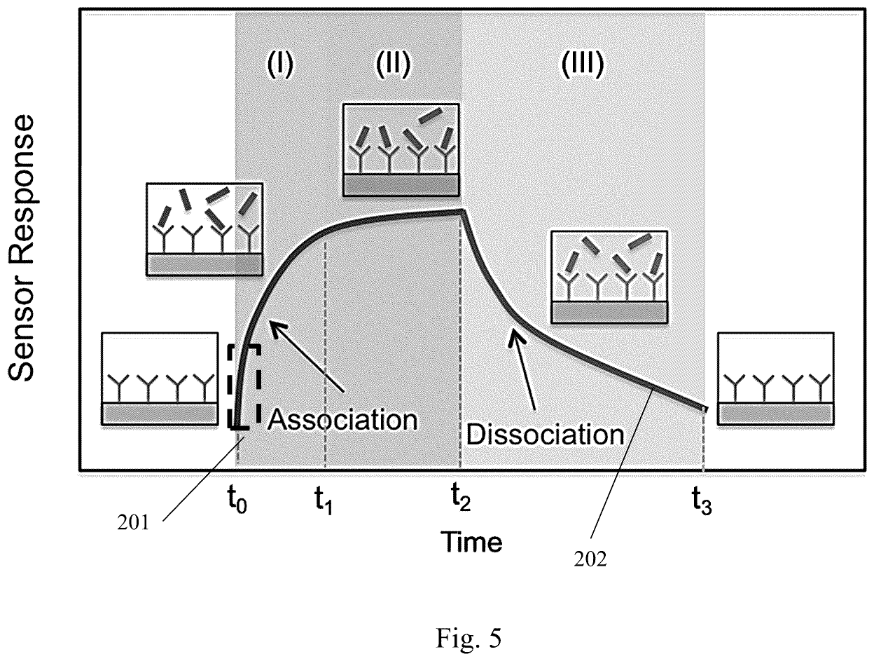

With reference to FIG. 5, a graph of the sensor response 202 over time is shown in a typical detection. In some embodiments, the sensor response is measured in Volts. The sensing moves from an initialization to a region, denoted by a (I), which occurs when the analytes 101 begin to combine with the surface probes 102. The first stage is defined by the transience to the steady state of a second region, denoted by (II). The dynamics can in some embodiments be determined using a two compartment model defined in Equation 1 below:

$$V \frac{{d [ A ]}_{s}}{dt} - S ( {k}_{m} ( [ A ] - {[ A ]}_{s} ) - {{k}_{1} [ A ]}_{s} ( {[ B ]}_{max} - [ AB ] ) + {k}_{- 1} [ AB ] ) \qquad Equation 1$$

where k₁ is the association rate constant, k₋₁ is the dissociation rate constant, and km is the effective transport rate constant. In such a scheme, the sensor response time is (t₁-t₀) and the maximum temporal resolution in Hz is (t₁-t₀)⁻¹.

Region II is defined as the steady state region, where traditional detection is performed. Once the signal measured is sufficiently constant, the sensor value is recorded. In this way, only one measurement is possible for determining the concentration of the analyte.

Finally, Region III is the dissociation region, which corresponds for example to a sensor wash, where analyte particles are removed from the sensor surface and the sensor is made ready for the next measurement.

A key finding of the present disclosure is that the rise time (shown with broken lines box 201) increases with reduced analyte concentration (and conversely decreases with increased analyte concentration). Therefore, a measurement of the rise time may be used to more quickly calculate the concentration. By adjusting the parameters of the appropriate circuitry described in a later embodiment, the detection time can be from a few seconds to sub-second. In some embodiments, the rise time measurement may be a less precise predictor, and so multiple measurements, either in parallel with multiple sensors, or sequential measurements with one sensor, may be used to increase precision. These two approaches themselves have drawbacks in a loss of spatial resolution and temporal resolution, respectively.

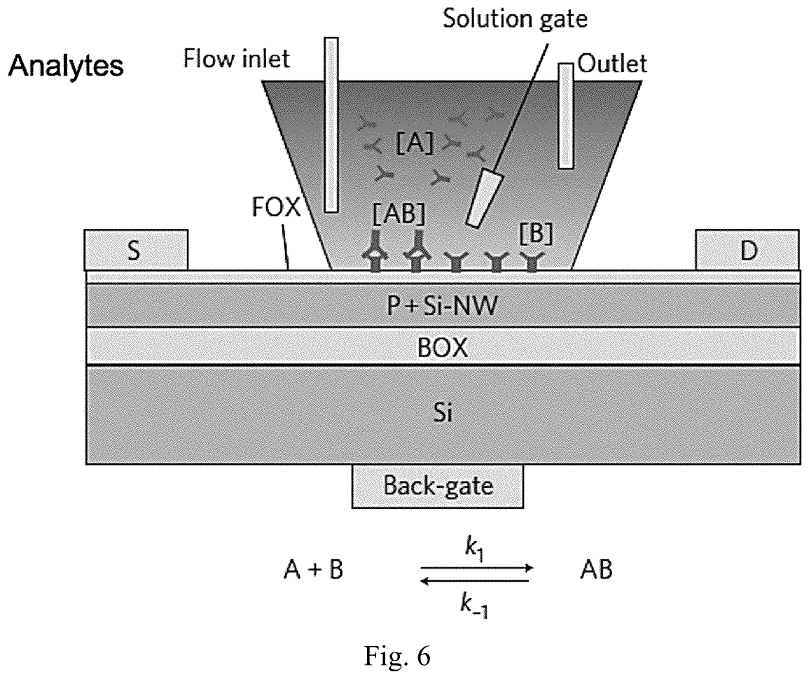

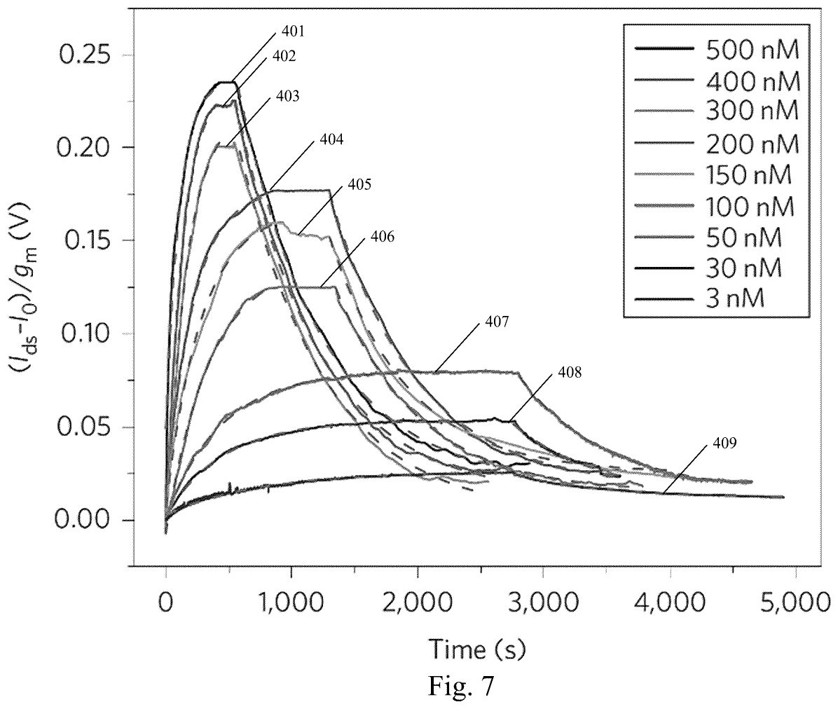

An example using a FET sensor is shown in FIG. 6 and FIG. 7, and further described in Nature Nanotechnology, Volume 7, pages 401-407 (2012); hereby incorporated herein by reference in its entirety. The exemplary FET sensor is shown in FIG. 6. FIG. 7 shows a graph of DNA+HMGB1 detection over time at a range of concentrations-curve 401 corresponds to 500 nM, curve 402 corresponds to 400 nM, curve 403 corresponds to 300 nM, curve 404 corresponds to 200 nM, curve 405 corresponds to 150 nM, curve 406 corresponds to 100 nM, curve 407 corresponds to 50 nM, curve 408 corresponds to 30 nM, and curve 409 corresponds to 3 nM. As shown, the temporal resolution at 3 nM is less than 0.33 Hz.

According to the two-compartment model, when the analyte/surface probe reaction is not mass transport limited (i.e. mass transport coefficient is large), the transience is an exponential function and depends on the association rate constant (k₁) and analyte concentration [A]. One aspect of the present disclosure is to use the fundamentals of the analyte/surface probe reaction kinetics for predicting the analyte concentration from the slope of transient curve. This principle is in some embodiments applicable to all sensing modalities and sensor types.

The disclosed method is advantageous because it improves temporal resolution, and also in some embodiments the disclosed measurement scheme can be applied multiple times during the transient time of the same “A+B” reaction (i.e., t₁-t₀ timescale). This allows averaging for reducing error and improving confidence.

One example is presented below with regard to a FET sensor. The output of a FET sensor, V(t), is given by Equation 2 below.

$$V ( t ) = \frac{{I}_{d s} ( t )}{{g}_{m}} = {V}_{e q} ( 1 - {e}^{- ( {k}_{1} [ A ] + {k}_{- 1} ) t} ) \qquad Equation 2$$

where V_{eq} is determined via calibration and is dependent on sensor type, and the steady-state value of the threshold voltage shift for a given analyte concentration [A] is given by Equation 3 below:

$${V}_{e q} = \frac{{q}_{A} [ {B}_{max} ]}{{C}_{0}} \frac{{k}_{1} [ A ]}{{k}_{1} [ A ] + {k}_{- 1}} \qquad Equation 3$$

### Expanding V(t) Using Taylor Series Yields Equation 4 Below:

$$V ( t ) = V ( 0 ) + {V}^{′} ( 0 ) t + Error ⇒ Δ V = \frac{{V}_{e q}}{τ} t ⇒ \frac{Δ {I}_{d s}}{{g}_{m}} = Δ V = \frac{{V}_{e q}}{τ} t = α [ A ] t \qquad Equation 4$$

where α and τ are given by Equation 5 and Equation 6, respectively.

$$τ = \frac{1}{{k}_{1} [ A ] + {k}_{- 1}} \qquad Equation 5$$

$$α = \frac{{q}_{A} [ {B}_{max} ]}{{C}_{0}} {k}_{1} \qquad Equation 6$$

Differentiation of V(t) can then be used to determine concentration based on the slope of the transient (rise time portion, region I) of the sensor response curve.

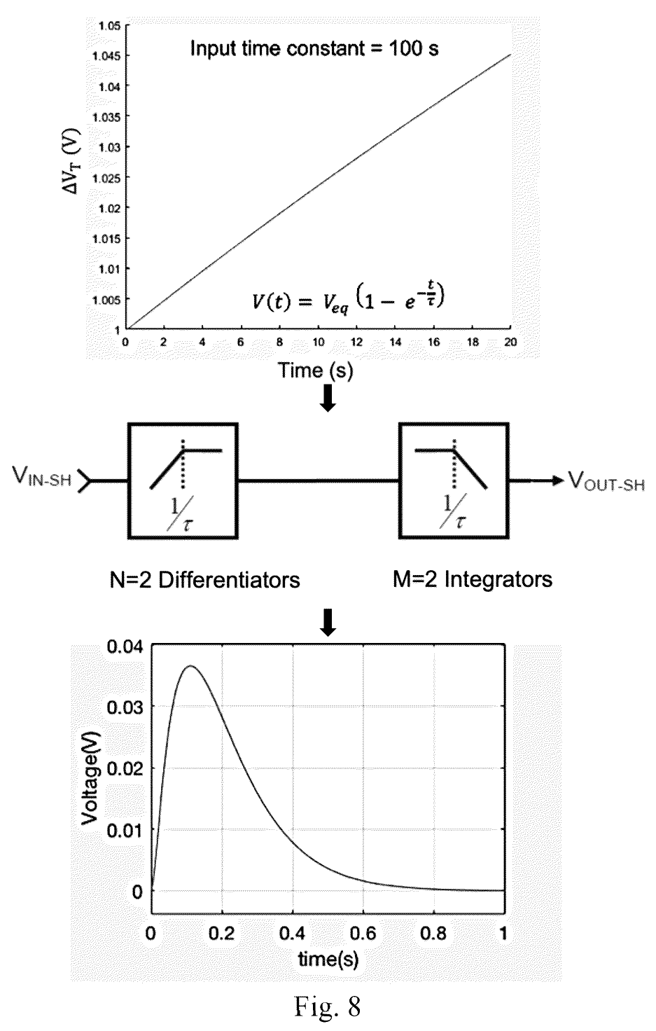

In some embodiments, pulse shaping circuits may be used to condition the sensor output, for example as shown in FIG. 8. In some embodiments, the pulse shaping circuits are designed to not be sensitive to noise in the sensor response, particularly in Region I from t₀ to t₁, and/or initial sensor response region 201. In some embodiments, (CR)ⁿ-(RC)^{m} pulse shaping circuits are used. These circuits are used in particle detectors for high-energy physics (which have timescale of μs). Such circuits may be configured for use in pulse-shaping of signals with time constants of 1 to 1000 sec⁻¹. In some embodiments, to accommodates such slow signals, the time constant of the high-pass and low-pass filters are adjusted accordingly, and large resistors in the Ohm range are used. In some embodiments, to permit the on-chip integration of these circuits, pseudo-resistors were used to achieve the desired time constants. In some embodiments, m=n=2. In some embodiments, m and/or n are less than or equal to 4.

In some embodiments, a front-end sensing stage of the detection circuitry comprises a differential amplifier pair of biosensors. In some embodiments, the output signal of the front-end sensing stage feeds the signal chain of the pulse shaping circuitry. In some embodiments, one biosensor of the differential amplifier pair has capturing probes on its surface. In some embodiments, one biosensor of the differential amplifier pair does not have capturing probes on its surface. In some embodiments, the differential amplifier pair of biosensors cancels the electrical signals due to non-specific binding. In some embodiments, the differential amplifier pair of biosensors cancels the environmental noise. In some embodiments, the pulse shaping detection circuit comprises a plurality of amplification stages along a signal chain. In some embodiments, the front-end biosensing stage is a single-stage amplifier. In some embodiments, the output of the front-end biosensing stage feeds a signal chain of the pulse shaping circuitry. In some embodiments, the biosensor of the front-end amplifier has capturing probes on its surface. In some embodiments, an electrical signal output of the pulse shaping detection circuit is the predictor of the analyte concentration. In some embodiments, the amplitude of the output of the pulse shaping detection circuit is proportional to the slope of the electrical output signal of the front-end biosensing stage.

Assuming a shaper architecture of (CR)²—(RC)², an analytical model for predicting the peak value of the voltage output of the shaper circuit is given in Equation 7 and Equation 8 below. Equation 7 is for a (CR)²—(RC)¹ shaper circuit, and Equation 8 is for a (CR)²—(RC)² shaper circuit.

$$Peak = \frac{{V}_{eq}}{τ} \times ( G \times {e}^{- \frac{{P}_{L}}{( {P}_{L} - {P}_{H} )}} \times \frac{{P}_{L}}{{P}_{H} ( {P}_{L} - {P}_{H} )} ) \qquad Equation 7$$

$$Peak = \frac{{V}_{eq}}{τ} ( {e}^{- \frac{{P}_{L} + {P}_{H}}{{P}_{L} - {P}_{H}}} \frac{{GP}_{L}^{2}}{{{P}_{H} ( {P}_{L} - {P}_{H} )}^{2}} ) \qquad Equation 8$$

An exemplary shaper circuit for use with the disclosed system is shown in FIG. 9A, with detail views shown as follows: current generator in FIG. 9B, two first order high-pass filters shown in FIG. 9C, second order low pass filters shown in FIG. 9D, and a gain stage shown in FIG. 9E. In some embodiments, all the GΩ resistors (shown in white ovals) are replaced with active resistors or pseudo-resistors. In some embodiments, the gain stage is realized by a PGA (programmable-gain amplifier).

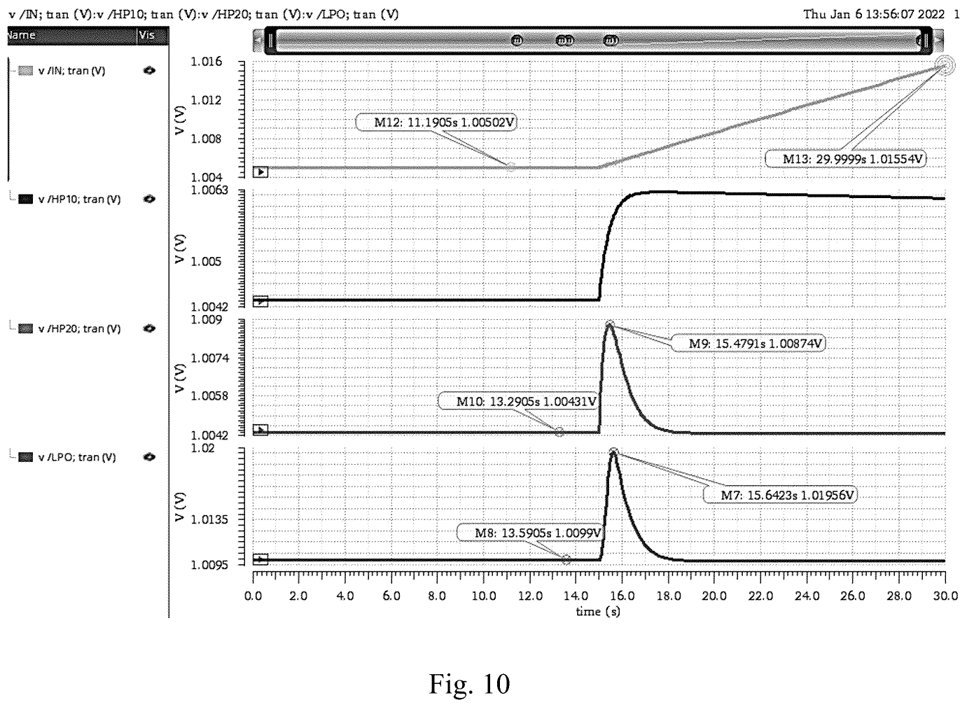

Results of simulations using the shaper circuit of FIG. 9A are shown in FIG. 10, where τ_{L}=0.1 s & τ_{H}=0.3 s.

In some embodiments, an analytical model utilizing pole values P_{L} and P_{H} of the low and high pass filters, respectively, is used to determine peak values. In some embodiments, the determined peak values are correlated to the concentration once calibrated, and can thus be used to determine the concentration. In some embodiments, according to Equation 8, peak amplitude is inversely proportional to τ, where τ (according to Equation 5) depends on k₁, k₋₁, and [A].

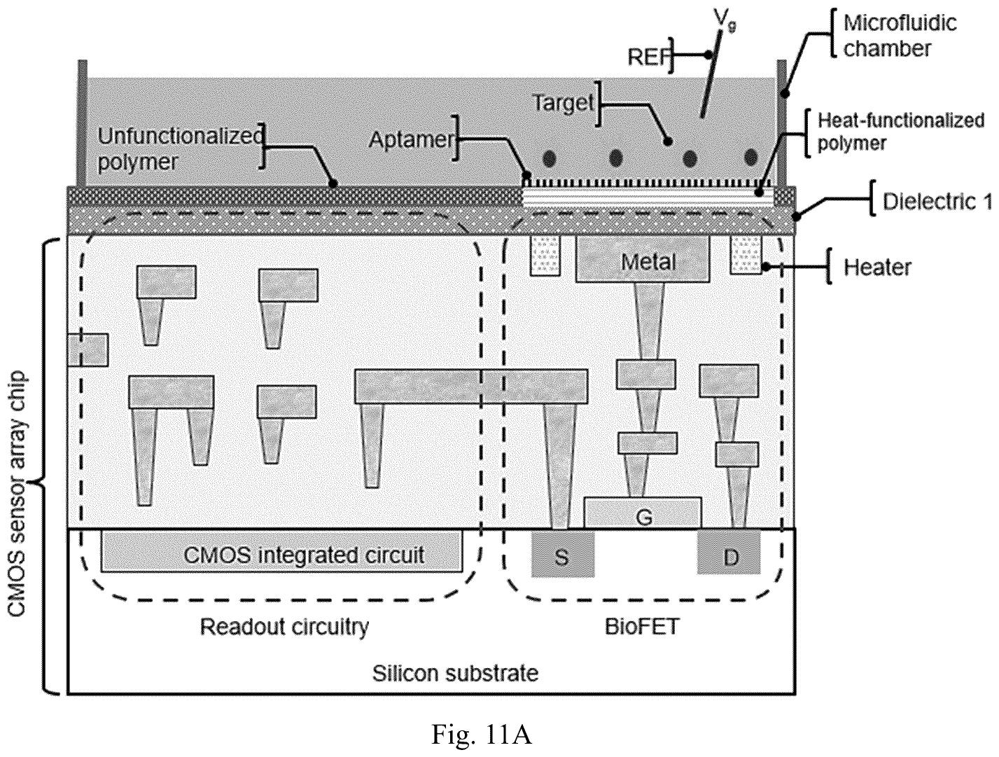

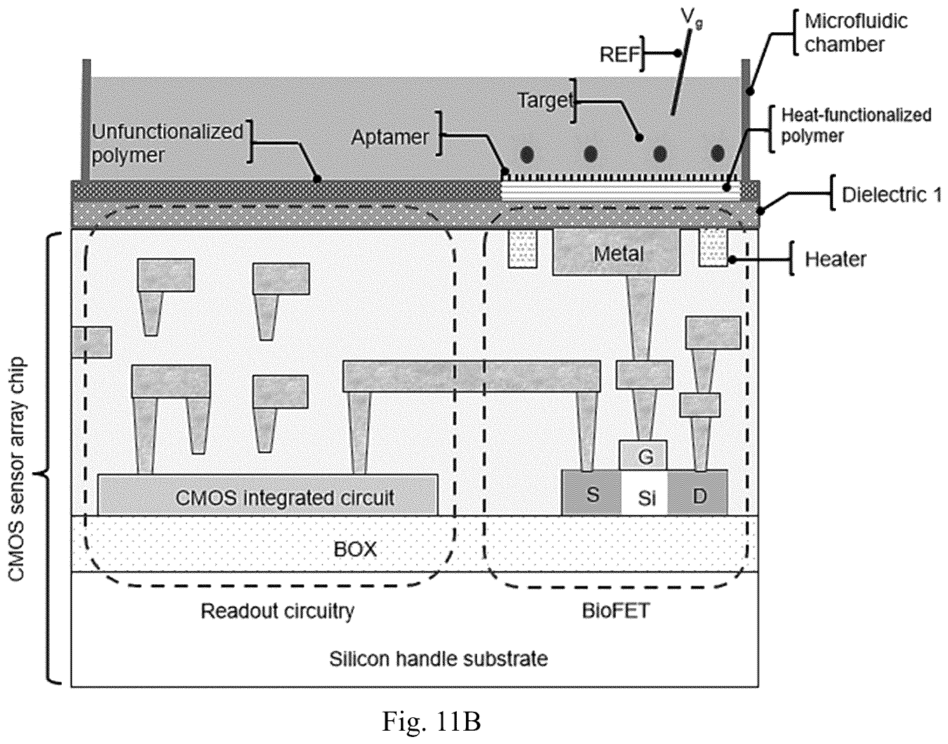

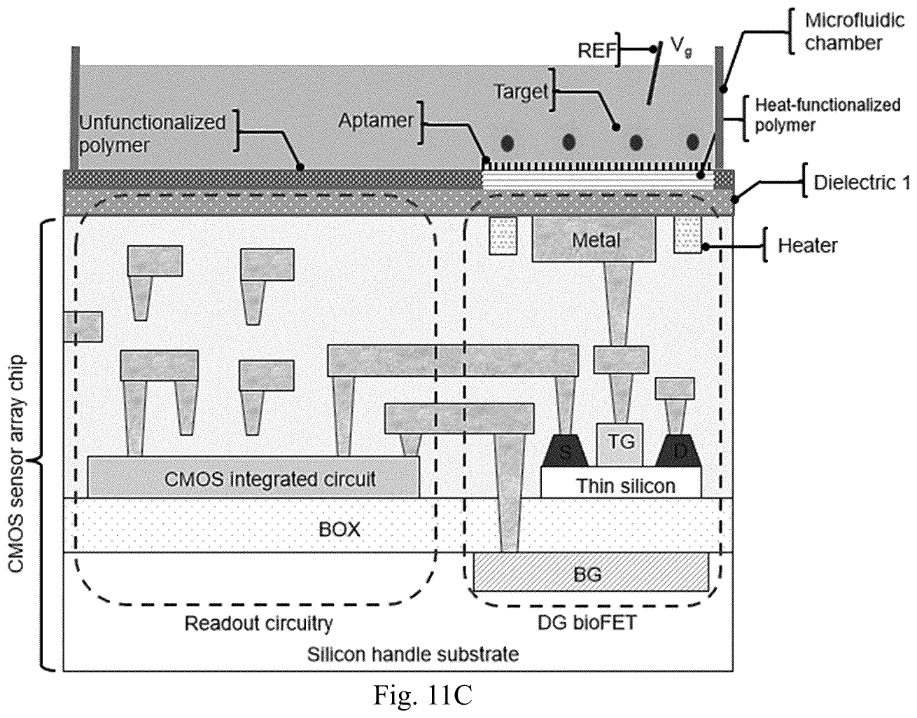

Three exemplary structures with biofunFETs monolithically integrated with CMOS readout circuitry are shown in FIG. 11A, FIG. 11B, and FIG. 11C. FIG. 11A shows a Bulk CMOS sensing platform, FIG. 11B shows a SOI CMOS sensing platform, and FIG. 11C shows an FDSOI CMOS sensing platform.

In FIG. 11A, FIG. 11B, and FIG. 11C, the letter “S” denotes a source, the letter “D” denotes a drain, the letters “TG” denote a top gate, the letters “BG” denote a bottom gate, the letter “G” denotes a gate, and the letters “DG” denote a double gate. “Sensing Pixel” refers to the biofunFET+the Readout Circuitry. “SOI” refers to silicon-on-insulator. The “Target” is the target molecule, virus, etc. “BOX” refers to a buried oxide. Vg is a gate bias voltage. REF is a reference electrode.

In the various embodiments of FIG. 11A, FIG. 11B, and FIG. 11C, biofunFETs may have a single-gate for SOI and bulk CMOS technologies. BiofunFETs can operate in single- or double-gate configurations in FDSOI technology. Monolithically, biofunFETs are made of silicon in these structures. BiofunFETs may in some embodiments be heterogeneously integrated on top of fully fabricated CMOS chips.

In some structures, biofunETs can be made of different functional semiconductors. Moreover, heterogeneously integrated biofunFETs can operate in single- or double-gate configurations. In some disclosed structures, a polymer positioned over the sensing surface may be functionalized locally using CMOS-integrated heaters or external sources of heat (e.g., thermal scanning tip, laser, etc).

### System

In one embodiment, the invention provides systems comprising the biofunFET, CMOS chip, or a combination thereof, of the invention. In some embodiments, the system comprises a computing device. The computing device may include a desktop computer, laptop computer, tablet, smartphone or other device and includes a software platform for control of the system components, display of raw data, and analysis of acquired data. The computing devices may include at least one processor, standard input and output devices, as well as all hardware and software typically found on computing devices for storing data and running programs, and for sending and receiving data over a network.

In certain embodiments, the system of the invention comprises hardware and software which detect and quantify detection signals from the biofunFET or the CMOS chip. The signals may be quantified using any suitable analysis software package, or using custom made analysis algorithms.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

### REFERENCES

The following publications are incorporated herein by reference in their entirety.
X. Liu, et al., Sub-10 nm Resolution Patterning of Pockets for Enzyme Immobilization with Independent Density and Quasi-3D Topography Control, ACS Applied Materials and Interfaces, 11, 2019
X. Liu, et al., Cost and Time Effective Lithography of Reusable Millimeter Size Bone Tissue Replicas With Sub-15 nm Feature Size on A Biocompatible Polymer, Adv. Func. Mater. 2021
D. Wang, et al., Thermochemical Nanolithography of Multifunctional Nanotemplates for Assembling Nano-Objects, Adv. Func. Mater., 2009
T. Wu, et al., Experimental Study of the Detection Limit in Dual-Gate Biosensors Using Ultrathin Silicon Transistors, ACS Nano, 2017
E. Cuniberto, et al., Nano-engineering the material structure of preferentially oriented nano-graphitic carbon for making high-performance electrochemical micro-sensors, Scientific Reports, 2020
X. Zheng, et al., Patterning metal contacts on monolayer MoS₂ with vanishing Schottky barriers using thermal nanolithography, Nature Electronics, 2019

What is claimed is:

1. A method of measuring a concentration of a target analyte in a volume, comprising:
providing a biosensor;
exposing the biosensor to the volume;
periodically measuring a sensor response;
calculating a slope of the measured sensor response as the analytes bind to the capturing probes on the surface; and
calculating the concentration of the target analyte in the volume based on the slope of the measured sensor response.

2. The method of claim 1, wherein the biosensor comprises a sensor surface and at least one surface capturing probe.

3. The method of claim 1, wherein the step of calculating a slope of the measured sensor response comprises calculating a slope of the measured sensor response during the binding phase a plurality of times, and averaging the calculated slope.

4. The method of claim 1, wherein the biosensor comprises a pulse shaping circuit configured to increase the temporal resolution of the sensor.

5. The method of claim 1, wherein the biosensor comprises a pulse shaping circuit configured to reduce noise in the sensor response during the binding phase.

6. The method of claim 5, wherein the pulse shaping circuit comprises a (CR)ⁿ-(RC)^{m} circuit.

7. The method of claim 6, wherein the (CR)ⁿ-(RC)^{m} circuit is configured for use in pulse-shaping of signals with time constants of 1 to 1000 sec⁻¹.

8. The method of claim 6, wherein the (CR)ⁿ-(RC)^{m} circuit comprises a (CR)²—(RC)² circuit.

9. The method of claim 1, wherein the step of calculating a slope of the measured sensor response comprises calibrating based on the output of a pulse shaping detection circuitry.

10. The method of claim 1, further comprising calibrating the biosensor for quantifying association and dissociation rate constants.

11. A system for measuring a concentration of a target analyte in a volume, comprising:
a biosensor having a sensing surface and an output terminal, the output terminal configured to change electrical signal based on the response from the sensing surface; and
a pulse shaping detection circuit configured to increase temporal resolution of sensing and to reduce noise in the sensor response during a binding phase.

12. The system of claim 11, wherein the pulse shaping detection circuit comprises two first order high-pass filters connected in a series having an input and an output, having the input of the series electrically connected to the output terminal.

13. The system of claim 12, wherein the pulse shaping detection circuit comprises a second order low-pass filter having an input and an output, the input of the second order low-pass filter connected to the output of the series.

14. The system of claim 13, wherein the pulse shaping detection circuit comprises a gain stage having an input and an output, the input of the gain stage connected to the output of the second order low-pass filter.

15. The system of claim 11, wherein the pulse shaping detection circuit comprises a (CR)ⁿ—(RC)^{m} circuit.

16. The system of claim 11, wherein the (CR)ⁿ-(RC)^{m} circuit is configured for use in pulse-shaping of signals with time constants of 1 to 1000 sec⁻¹.

17. The system of claim 11, wherein the (CR)ⁿ-(RC)^{m} circuit comprises a (CR)²—(RC)² circuit.

18. The system of claim 11, wherein a front-end sensing stage of the pulse shaping detection circuit comprises a differential amplifier pair of biosensors.

19. The system of claim 18, wherein the output signal of the front-end sensing stage feeds a signal chain of the pulse shaping detection circuit.

20. The system of claim 18, wherein one biosensor of the differential amplifier pair has capturing probes on its surface.

21. The system of claim 18, wherein one biosensor of the differential amplifier pair does not have capturing probes on its surface.

22. The system of claim 18, wherein the differential amplifier pair of biosensors cancels the electrical signals due to non-specific binding.

23. The system of claim 18, wherein the differential amplifier pair of biosensors cancels the environmental noise.

24. The system of claim 11, wherein the pulse shaping detection circuit comprises a plurality of amplification stages along a signal chain.

25. The system of claim 18, wherein the front-end biosensing stage is a single-stage amplifier.

26. The system of claim 18, wherein the output of the front-end biosensing stage feeds a signal chain of the pulse shaping circuitry.

27. The system of claim 25, wherein the biosensor of the front-end amplifier has capturing probes on its surface.

28. The system of claim 11, wherein an electrical signal output of the pulse shaping detection circuit is the predictor of the analyte concentration.

29. The system of claim 28, wherein the amplitude of the output of the pulse shaping detection circuit is proportional to the slope of the electrical output signal of the front-end biosensing stage.
